(19) United States
(12) Patent Application Publication
(10) Pub. No.: US 2024/0307005 A1
(43) Pub. Date: Sep. 19, 2024

(54) Systems and Processes for Noninvasive Blood Pressure Estimation

(71) Applicant: Worcester Polytechnic Institute, Worcester, MA (US)

(72) Inventors: Rajesh S. Kasbekar, Shrewsbury, MA (US); Edward A. Clancy, Chelmsford, MA (US)

(73) Assignee: Worcester Polytechnic Institute, Worcester, MA (US)

(21) Appl. No.: 18/605,287

(22) Filed: Mar. 14, 2024

Related U.S. Application Data
(60) Provisional application No. 63/452,007, filed on Mar. 14, 2023.

(51) Int. Cl. A61B 5/00; A61B 5/021; A61B 5/352; G16H 10/60; G16H 50/70

(52) U.S. Cl. CPC A61B 5/7267; A61B 5/02125; A61B 5/352; A61B 5/681; G16H 10/60; G16H 50/70

(57) ABSTRACT

This disclosure relates to systems and processes for estimating blood pressure. Systems described herein comprise at least one sensor for measuring at least one waveform related to blood pressure from a patient; at least one processor; and at least one computer-readable storage medium having encoded thereon executable instructions to carry out a method comprising: receiving, from the at least one sensor, the at least one waveform and determining one or more features comprising at least one or more temporal features and one or more morphology features; and analyzing the one or more features using one or more trained models to determine one or more blood pressure (BP) value associated with the patient; and outputting the one or more BP value determined for the patient based on the received one or more features. The system allows for continuous and noninvasive calculations of a blood pressure to the patient or other users.

### CROSS-REFERENCE

This application claims priority to and the benefit of U.S. Provisional Application Ser. No. 63/452,007 filed on Mar. 14, 2023, which is incorporated herein by reference in its entirety for all purposes.

### BACKGROUND

### Field

This disclosure relates to systems and methods for measuring blood pressure (BP), and, in particular, to an integrated BP estimation system included in a cuffless, wearable device that can provide continuous and noninvasive calculations of BP to the patient or other users.

### Description

Hypertension remains a leading risk factor for death and quality of life in both high- and low-income countries. Detection, monitoring and control, however, are inadequate, with only 46% of individuals with hypertension having their condition under control. The complications of hypertension are responsible for premature deaths worldwide.

Traditionally, BP is estimated using a cuff-based monitor. However, cuff-based methods have proven to be a barrier to widespread use of BP measurement due to discomfort associated with measurements, intermittent measurements and the bulky equipment that is needed. A simple and user-friendly device that also measures continuous diurnal BP would better enable hypertension management, as well as the management of many other diseases.

### SUMMARY

This section includes a summary of the claims in the commonly accepted definition of a comprehensive and usually brief recapitulation of the claims. It should be appreciated that these embodiments are merely illustrative, that embodiments are not limited to operating in accordance with the specific examples shown in the figures and discussed below, and that other embodiments are possible. The following embodiments are put forth so as to provide those of ordinary skill in the art with a complete disclosure and description of how to make and use the blood pressure estimating systems and methods of use and manufacture as described in some aspects and embodiments herein, and are not intended to limit the scope of the various aspects and embodiments herein.

In some aspects, the techniques described herein relate to a system for estimating blood pressure including: at least one sensor configured to measure at least one waveform related to blood pressure from a patient; at least one processor; and at least one computer-readable storage medium having encoded thereon executable instructions that, when executed by the at least one processor, cause the at least one processor to carry out a method including: (a) receiving, from the at least one sensor, the at least one waveform and determining, from the at least one waveform, one or more features including at least one or more temporal features and one or more morphology features; (b) analyzing the one or more features using one or more trained models to determine one or more blood pressure (BP) value associated with the patient; and (c) outputting the one or more BP value determined for the patient based on the received one or more features.

In some aspects, the techniques described herein relate to a system, further including a user interface.

In some aspects, the techniques described herein relate to a system, wherein at least one of the at least one sensor, the at least one processor, and the at least one computer-readable storage medium is wearable.

In some aspects, the techniques described herein relate to a system, wherein the at least one sensor includes a photoplethysmography (PPG) sensor or an electrocardiogram (ECG) sensor.

In some aspects, the techniques described herein relate to a system, wherein each of the one or more BP value includes one or more of a systolic BP, a diastolic BP, and/or a mean arterial pressure (MAP).

In some aspects, the techniques described herein relate to a system, wherein the received one or more features is a preprocessed feature set, the preprocessed feature set including the one or more features configured as a function of one or more of: a pre-ejection period, a square of pulse transit time, a PPG intensity ratio, and/or a Womersly number.

In some aspects, the techniques described herein relate to a system, wherein the one or more trained models includes one or more of: a lasso model, a random forest model, a support vector machine model, an artificial neural network model, a long short term memory model, a RESNET deep learning model, and/or a combination or ensemble thereof.

In some aspects, the techniques described herein relate to a system, wherein the analyzing includes tuning hyperparameters for each of the one or more trained models.

In some aspects, the techniques described herein relate to a system, wherein the one or more morphology features include: a BP cycle time, an ejection time, an artery fill time, an artery emptying time, a peak volume, a systolic volume, a systolic volume differential, a diastolic volume, a diastolic volume differential, or a combination thereof.

In some aspects, the techniques described herein relate to a system, wherein the one or more morphology features are obtained from at least one PPG waveform, wherein the at least one PPG waveform is collected noninvasively over a radial artery.

In some aspects, the techniques described herein relate to a system, wherein the one or more features further includes biometric data, wherein the biometric data includes one or more of: a pre-existing condition, an age, a weight, a height, a waist size, a body mass index (BMI), a sex, and/or a combination thereof.

In some aspects, the techniques described herein relate to a system, wherein the one or more temporal features includes one or more of: a pulse arrival time (PAT), a pulse transit time (PTT), a pulse rate, and/or a combination thereof.

In some aspects, the techniques described herein relate to a system, wherein the PAT is based on a time difference between a peak of an ECG-R wave and a peak of a PPG waveform.

In some aspects, the techniques described herein relate to a system, wherein the PTT is based on a difference between at least two PPG waveforms.

In some aspects, the techniques described herein relate to a system, wherein at least one of the one or more temporal features and the one or more morphology features are extracted from: a first PPG sensor and a second PPG sensor, a first PPG sensor and a first ECG sensor, or a first ECG sensor and a second ECG sensor.

In some aspects, the techniques described herein relate to a system, wherein each of the one or more trained models are trained using at least training temporal feature data and training morphology data from a plurality of prior patients.

In some aspects, the techniques described herein relate to a system, wherein each of the prior patients has a pre-existing condition.

In some aspects, the techniques described herein relate to a system, wherein the determining includes selecting the one or more trained models from a plurality of models, wherein the selecting is based on evaluating a performance of the one or more trained models as compared to others of the plurality of models.

In some aspects, the techniques described herein relate to a system, wherein the evaluating is based on one or more of an average error bias and/or a standard deviation of each of the plurality of models.

In some aspects, the techniques described herein relate to a system, wherein the evaluating is performed independently for each of the one or more BP value.

In some aspects, the techniques described herein relate to a system, wherein the determining includes selecting an ensemble of two or more of the one or more trained models, wherein the selecting is based on evaluating a performance of the one or more trained models as compared to others of the one or more trained models.

In some aspects, the techniques described herein relate to a system, wherein the ensemble includes the two or more of the one or more trained models being in parallel or serial.

In some aspects, the techniques described herein relate to a method for estimating blood pressure, the method including: (a) determining, based on a received feature set of one or more features from a patient, one or more blood pressure (BP) value associated with the patient, the one or more features including at least one or more temporal features and one or more morphology features, the determining including analyzing the one or more features using one or more trained models, wherein each of the one or more trained models are trained using at least training temporal feature data and training morphology data from a plurality of prior patients; and (b) outputting the one or more BP value determined for the patient based on the received feature set.

In some aspects, the techniques described herein relate to a method, wherein the one or more BP value includes one or more of a systolic BP, a diastolic BP, and/or a mean arterial pressure (MAP).

In some aspects, the techniques described herein relate to a method, wherein the determining further includes preprocessing the received feature set prior to the analyzing, the preprocessing including configuring the one or more features as a function of one or more of: a pre-ejection period, a square of pulse transit time, a photoplethysmography (PPG) intensity ratio, and/or a Womersly number.

In some aspects, the techniques described herein relate to a method, wherein the one or more trained models includes one or more of: a lasso model, a random forest model, a support vector machine model, an artificial neural network model, a long short term memory model, a RESNET deep learning model, and/or a combination or ensemble thereof.

In some aspects, the techniques described herein relate to a method, wherein the analyzing includes tuning hyperparameters for each of the one or more trained models.

In some aspects, the techniques described herein relate to a method, wherein the one or more morphology features include: a BP cycle time, an ejection time, an artery fill time, an artery emptying time, a peak volume, a systolic volume, a systolic volume differential, a diastolic volume, a diastolic volume differential, or a combination thereof.

In some aspects, the techniques described herein relate to a method, wherein the one or more morphology features are obtained from at least one PPG waveform, wherein the at least one PPG waveform is collected noninvasively over a radial artery.

In some aspects, the techniques described herein relate to a method, wherein the feature set further includes biometric data, wherein the biometric data includes one or more of: a pre-existing condition, an age, a weight, a height, a waist size, a body mass index (BMI), a sex, and/or a combination thereof.

In some aspects, the techniques described herein relate to a method, wherein the one or more temporal features includes one or more of: a pulse arrival time (PAT), a pulse transit time (PTT), a pulse rate, and/or a combination thereof.

In some aspects, the techniques described herein relate to a method, wherein the PAT is based on a time difference between a peak of an ECG-R wave and a peak of a PPG waveform.

In some aspects, the techniques described herein relate to a method, wherein the PTT is based on a difference between at least two PPG waveforms.

In some aspects, the techniques described herein relate to a method, wherein at least one of the one or more temporal features and the one or more morphology features are extracted from: a first PPG sensor and a second PPG sensor, a first PPG sensor and a first electrocardiogram (ECG) sensor, or a first ECG sensor and a second ECG sensor.

In some aspects, the techniques described herein relate to a method, wherein each of the plurality of prior patients has a pre-existing condition.

In some aspects, the techniques described herein relate to a method, wherein the determining includes selecting the one or more trained models from a plurality of models, wherein the selecting is based on evaluating a performance of the one or more trained models as compared to others of the plurality of models.

In some aspects, the techniques described herein relate to a method, wherein the evaluating is based on one or more of an average error bias and/or a standard deviation of each of the plurality of models.

In some aspects, the techniques described herein relate to a method, wherein the evaluating is performed independently for each of the one or more BP value.

In some aspects, the techniques described herein relate to a method, wherein the determining includes selecting an ensemble of two or more of the one or more trained models, wherein the selecting is based on evaluating a performance of the one or more trained models as compared to others of the plurality of models.

In some aspects, the techniques described herein relate to a method, wherein the ensemble includes the two or more of the one or more trained models being in parallel or serial.

In some aspects, the techniques described herein relate to at least one storage medium having encoded thereon executable instructions that, when executed by at least one processor, cause the at least one processor to carry out a method including: (a) determining, based on a received feature set of one or more features from a patient, one or more blood pressure (BP) value associated with the patient, the one or more features including at least one or more temporal features and one or more morphology features, the determining including analyzing the one or more features using one or more trained models, wherein each of the one or more trained models are trained using at least training temporal feature data and training morphology data from a plurality of prior patients; and (b) outputting the one or more BP value determined for the patient based on the received feature set.

In some aspects, the techniques described herein relate to an at least one storage medium, wherein the one or more BP value includes one or more of a systolic BP, a diastolic BP, and/or a mean arterial pressure (MAP).

In some aspects, the techniques described herein relate to an at least one storage medium, wherein the determining further includes preprocessing the received feature set prior to the analyzing, the preprocessing including configuring the one or more features as a function of one or more of: a pre-ejection period, a square of pulse transit time, a photoplethysmography (PPG) intensity ratio, and/or a Womersly number.

In some aspects, the techniques described herein relate to an at least one storage medium, wherein the one or more trained models includes one or more of: a lasso model, a random forest model, a support vector machine model, an artificial neural network model, a long short term memory model, a RESNET deep learning model, and/or a combination or ensemble thereof.

In some aspects, the techniques described herein relate to an at least one storage medium, wherein the analyzing includes tuning hyperparameters for each of the one or more trained models.

In some aspects, the techniques described herein relate to an at least one storage medium, wherein the one or more morphology features include: a BP cycle time, an ejection time, an artery fill time, an artery emptying time, a peak volume, a systolic volume, a systolic volume differential, a diastolic volume, a diastolic volume differential, or a combination thereof.

In some aspects, the techniques described herein relate to an at least one storage medium, wherein the one or more morphology features are obtained from at least one PPG waveform, wherein the at least one PPG waveform is collected noninvasively over a radial artery.

In some aspects, the techniques described herein relate to an at least one storage medium, wherein the feature set further includes biometric data, wherein the biometric data includes one or more of: a pre-existing condition, an age, a weight, a height, a waist size, a body mass index (BMI), a sex, and/or a combination thereof.

In some aspects, the techniques described herein relate to an at least one storage medium, wherein the one or more temporal features includes one or more of: a pulse arrival time (PAT), a pulse transit time (PTT), a pulse rate, and/or a combination thereof.

In some aspects, the techniques described herein relate to an at least one storage medium, wherein the PAT is based on a time difference between a peak of an ECG-R wave and a peak of a PPG waveform.

In some aspects, the techniques described herein relate to an at least one storage medium, wherein the PTT is based on a difference between at least two PPG waveforms.

In some aspects, the techniques described herein relate to an at least one storage medium, wherein at least one of the one or more temporal features and the one or more morphology features are extracted from: a first PPG sensor and a second PPG sensor, a first PPG sensor and a first electrocardiogram (ECG) sensor, or a first ECG sensor and a second ECG sensor.

In some aspects, the techniques described herein relate to an at least one storage medium, wherein each of the plurality of prior patients has a pre-existing condition.

In some aspects, the techniques described herein relate to an at least one storage medium, wherein the determining includes selecting the one or more trained models from a plurality of models, wherein the selecting is based on evaluating a performance of the one or more trained models as compared to others of the plurality of models.

In some aspects, the techniques described herein relate to an at least one storage medium, wherein the evaluating is based on one or more of an average error bias and/or a standard deviation of each of the plurality of models.

In some aspects, the techniques described herein relate to an at least one storage medium, wherein the evaluating is performed independently for each of the one or more BP value.

In some aspects, the techniques described herein relate to an at least one storage medium, wherein the determining includes selecting an ensemble of two or more of the one or more trained models, wherein the selecting is based on evaluating a performance of the one or more trained models as compared to others of the plurality of models.

In some aspects, the techniques described herein relate to an at least one storage medium, wherein the ensemble includes the two or more of the one or more trained models being in parallel or serial.

In some aspects, the techniques described herein relate to a system for estimating blood pressure including: at least one processor; and at least one computer-readable storage medium having encoded thereon executable instructions that, when executed by the at least one processor, cause the at least one processor to carry out a method including: determining, based on a received feature set of one or more features from a patient, one or more blood pressure (BP) value associated with the patient, the one or more features including at least one or more temporal features and one or more morphology features, the determining including analyzing the one or more features using one or more trained models, wherein each of the one or more trained models are trained using at least training temporal feature data and training morphology data from a plurality of prior patients.

In some aspects, the techniques described herein relate to a system, further including a user interface.

In some aspects, the techniques described herein relate to a system, further including at least one sensor.

In some aspects, the techniques described herein relate to a system, wherein at least one of the at least one sensor, the at least one processor, and the at least one computer-readable storage medium is wearable.

In some aspects, the techniques described herein relate to a system, wherein the at least one sensor includes a photoplethysmography (PPG) sensor or an electrocardiogram (ECG) sensor.

In some aspects, the techniques described herein relate to a system, wherein the one or more BP value includes one or more of a systolic BP, a diastolic BP, and/or a mean arterial pressure (MAP).

In some aspects, the techniques described herein relate to a system, wherein the received one or more features is a preprocessed feature set, the preprocessed feature set including the one or more features configured as a function of one or more of: a pre-ejection period, a square of pulse transit time, a PPG intensity ratio, and/or a Womersly number.

In some aspects, the techniques described herein relate to a system, wherein the one or more trained models includes one or more of: a lasso model, a random forest model, a support vector machine model, an artificial neural network model, a long short term memory model, a RESNET deep learning model, and/or a combination or ensemble thereof.

In some aspects, the techniques described herein relate to a system, wherein the analyzing includes tuning hyperparameters for each of the one or more trained models.

In some aspects, the techniques described herein relate to a system, wherein the one or more morphology features include: a BP cycle time, an ejection time, an artery fill time, an artery emptying time, a peak volume, a systolic volume, a systolic volume differential, a diastolic volume, a diastolic volume differential, or a combination thereof.

In some aspects, the techniques described herein relate to a system, wherein the one or more morphology features are obtained from at least one PPG waveform, wherein the at least one PPG waveform is collected noninvasively over a radial artery.

In some aspects, the techniques described herein relate to a system, wherein the one or more features further includes biometric data, wherein the biometric data includes one or more of: a pre-existing condition, an age, a weight, a height, a waist size, a body mass index (BMI), a sex, and/or a combination thereof.

In some aspects, the techniques described herein relate to a system, wherein the one or more temporal features includes one or more of: a pulse arrival time (PAT), a pulse transit time (PTT), a pulse rate, and/or a combination thereof.

In some aspects, the techniques described herein relate to a system, wherein the PAT is based on a time difference between a peak of an ECG-R wave and a peak of a PPG waveform.

In some aspects, the techniques described herein relate to a system, wherein the PTT is based on a difference between at least two PPG waveforms.

In some aspects, the techniques described herein relate to a system, wherein at least one of the one or more temporal features and the one or more morphology features are extracted from: a first PPG sensor and a second PPG sensor, a first PPG sensor and a first ECG sensor, or a first ECG sensor and a second ECG sensor.

In some aspects, the techniques described herein relate to a system, wherein each of the one or more trained models are trained using at least training temporal feature data and training morphology data from a plurality of prior patients.

In some aspects, the techniques described herein relate to a system, wherein each of the plurality of prior patients has a pre-existing condition.

In some aspects, the techniques described herein relate to a system, wherein the determining includes selecting the one or more trained models from a plurality of models, wherein the selecting is based on evaluating a performance of the one or more trained models as compared to others of the plurality of models.

In some aspects, the techniques described herein relate to a system, wherein the evaluating is based on one or more of an average error bias and/or a standard deviation of each of the plurality of models.

In some aspects, the techniques described herein relate to a system, wherein the evaluating is performed independently for each of the one or more BP value.

In some aspects, the techniques described herein relate to a system, wherein the determining includes selecting an ensemble of two or more of the one or more trained models, wherein the selecting is based on evaluating a performance of the one or more trained models as compared to others of the plurality of models.

In some aspects, the techniques described herein relate to a system, wherein the ensemble includes the two or more of the one or more trained models being in parallel or serial.

Additional and/or other aspects and advantages of the present invention will be set forth in the description that follows, or will be apparent from the description, or may be learned by practice of the invention. The present invention may comprise an integrated BPE system and related devices and software apps and methods for operating same having one or more of the above aspects, and/or one or more of the features and combinations thereof. The present invention may comprise one or more of the features and/or combinations of the above aspects as recited, for example, in the attached claims.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 is a block diagram illustrating a system according to one embodiment;

FIGS. 2A-2C are flowcharts illustrating exemplary processes for a system according to an embodiment of this disclosure;

FIG. 3A is a block diagram illustrating a system according to one embodiment;

FIG. 3B is an exemplary implementation of a computing device that may be used in a system implementing techniques described herein;

FIG. 4 is an exemplary experimental setup for data collection; and

FIGS. 5A-5C are estimated blood pressure in a test dataset using combined feature datasets from hemodynamically compromised patients (n=126). Lines show ±2 standard deviations of all five algorithms. FIG. 5A provides mean arterial pressure (MAP) comparison, FIG. 5B provides systolic BP comparison, and FIG. 5C provides diastolic BP comparison. Axis scales differ between plots.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

This disclosure relates to blood pressure estimation (BPE) systems and methods that are continuous, cuffless, and non-invasive. The BPE systems and methods can include or use one or more trained model or an ensemble thereof using a combination of temporal, morphological and biometric features to aid a patient in managing his or her disease or illness. In some embodiments, BPE systems and methods receive information about the user from one or more user devices, such as internet-enabled user devices (e.g., a smartphone) or other smart or connected devices (such as patient monitors, fitness trackers, etc.). The information received from these devices can be used to customize the BPE systems and methods for the user, providing an efficient and effective means for managing the illness or disease. In certain embodiments, the illness or disease is a hypertension related disease such as cardiac morbidity, chronic renal failure, malignant and secondary hypertension, pre-eclampsia and conditions associated with autonomic neuropathy. The BPE system can lead to simple, quick, and readily available counseling regarding a healthy lifestyle.

There are three measures of BP that are powerful predictors of hypertension: average or true level, diurnal variation and short-term variability. Most clinical and epidemiologic data are available for the average or true level. However, there is significant diurnal variation in certain hypertensive subjects with chronic renal failure, malignant and secondary hypertension, pre-eclampsia and conditions associated with autonomic neuropathy. Cardiovascular morbidity and mortality have been shown to be linked to nighttime BP compared to daytime BP. Increased ambulatory BP variability has also been shown to be correlated with the development of early carotid arteriosclerosis and a high rate of cardiovascular morbidity. There is not sufficient evidence to apply these conclusions to routine clinical practice; however, the significance of the prognostic value of daytime and nighttime BP variability on cardiovascular events cannot be ignored.

Traditionally, BP is estimated using a cuff-based monitor. However, cuff-based methods have proven to be a barrier to widespread use of BP measurement due to its intermittent nature and the discomfort associated with measurements and the bulky equipment that is needed. A simple and user-friendly device that also measures continuous diurnal BP would better enable hypertension management. Continuous or ambulatory BP monitoring would also provide additional information than the information that is captured from intermittent clinical or home-use BP measurements.

For routine monitoring, diagnosis and treatment, arterial blood pressure (BP) is considered a universal indicator of hypertension and cardiovascular health. The gold standard for BP measurements in hospital and professional settings is intra-arterial catheterization, a method that is invasive and requires a sensor such as a strain gage to be in fluid contact with blood at an arterial site. The noninvasive method used in clinics is the auscultation-based BP cuff with mercury manometer method, where the brachial artery is occluded with a cuff placed around the upper arm inflated above the systolic BP, and Korotkoff sounds are detected using a stethescope during cuff deflation. Both of these methods have been used as a gold standard for decades with little innovation. However, patient discomfort, reduced patient mobility and intermittent monitoring are some of the limitations of this methodology. In addition, the effects of posture, body position, white coat hypertension and cuff size can significantly affect the accuracy of the readings, making this an unreliable method for measuring BP.

An automated BP cuff employing oscillometry can be used to measure BP in clinical and home settings. A pressure sensor in the cuff measures arterial pulsations during cuff deflation/inflation, and the pulse amplitudes are used to empirically derive systolic and diastolic BP. These algorithms are specific to the device and the cuff. If the pulse amplitude is weak, it can result in inaccurate readings, such as in obese people or for someone who has atheroschlerotic plaques. In addition, the application of pressure during cuff inflation/deflation can alter BP, resulting in a corrupted measurement.

Such automated cuff-based methods, whether used in the clinic or at home, have an inherent accuracy requirement of ±5 mm Hg bias and ±8 mm Hg standard deviation as a key criterion in the AAMI ISO-80601-2-30:2018 automated non-invasive sphygmomanometers standard. Wearing a cuff can be uncomfortable for elderly, diseased or handicapped subjects. It has resulted in dizziness and fainting in some instances. To address this issue and increase user friendliness, more comfortable cuffs for the wrist have been developed. These devices, however, have significantly lower measurement accuracy than upper arm BP monitors.

Other techniques used for intermittent BP monitoring include the ultrasound technique or the finger cuff method. In the ultrasound method, an ultrasound transmitter and receiver placed under a cuff detect a Doppler phase shift during the diastolic phase. The finger cuff method works on the principle of loading the arterial wall. In this method, arterial pulsation, as detected by a photoplethysmogram (PPG), is used to apply cuff pressure to the finger to keep the cardiac output constant. The resulting oscillations resemble intraarterial pressure waves with measurable morphological features, and this method gives systolic and diastolic pressure that is calibrated using an arm cuff. This method can be used to measure continuous BP but is expensive, inconvenient, highly variable based on its location as well as thermal characteristics of the finger, and limited in its application for hypotensive patients such that it is not suitable for consumer use.

Due to the limitations of methods requiring external pressure to the arteries in the arm or wrist, techniques for continuous BP monitoring have become popular. In addition, continuous BP monitoring has significant advantages compared to clinical BP taken in an office setting or an intermittent self-measured BP taken at home. With the advent of smartphones and wearables, continuous monitoring of BP has become more feasible. Specifically, continuous monitoring in BP telemonitoring could provide several benefits, such as generating more data, reducing patient discomfort, increasing the efficacy of lowering systolic and diastolic BP than traditional office-based care.

Various ML algorithms using classical and deep learning techniques have been utilized on measures associated with BP, such as PPG, bioimpedance, pressure tonography or other waveforms as well as pulse rate and other biometric data. In particular, several ML techniques have been used to predict systolic pressure, diastolic pressure and mean arterial pressure (MAP) by using morphology features extracted from PPG signals. However, such techniques have mostly been used to build models based on data from healthy patients, and have resulted in high mean errors preventing suitability in clinical applications.

BP regulation in arterial vessels is a complex phenomenon and is affected by many variables, including blood viscosity, blood thickness, stiffness and cross-sectional area of the blood vessels, and hormonal changes resulting in smooth muscle contraction. The inventors have recognized and appreciated that there is a strong association between BP and the velocity of the pulse wave that propagates through the arterial network. Thus, in addition to morphological features discussed above, an approach to noninvasive continuous estimation of systolic and diastolic BP may be based on measurement of temporal features. Such temporal features may include the pulse arrival time (PAT) or the pulse transit time (PTT). However, while such temporal features are good predictors of BP, they also vary with arterial compliance as well as arterial cross-sectional area, stroke volume or pre-ejection period (PEP), blood viscosity and blood thickness (Womersley number), cross-sectional area and thickness of the arterial wall (PPG Intensity ratio or PIR), and hormonal changes, all of which can change with time. Thus, the inventors have recognized and appreciated that the BP can be summarized as a function of all these factors as follows: BP=f (PEP, PAT, PIR, Womersley number). These changes are confounding factors in the accurate estimation of BP.

Features derived from pulse transit or arrival time and pulse wave morphology extracted from photoplethysmography (PPG) signals are increasingly used for continuous noninvasive blood pressure (BP) monitoring. The inventors have further recognized and appreciated that, through demonstration provided herein, computationally combining them for training provides a comprehensive model that takes into account all factors affecting BP instead of partial factors as identified by others, resulting in a higher accuracy and robustness for measuring BP.

In some embodiments, this disclosure provides (1) continuous BPE computational systems and processes that combine the calculations of both measured temporal and morphological features, and (2) data feature extraction processes for providing data to the continuous BPE computational module.

### Continuous BPE Computational Systems and Processes

Provided herein are systems and methods for estimating continuous BP measurements using at least one feature set comprising at least temporal features and morphological features. In some embodiments, the temporal features and morphological features are obtained from a user via extracted from each of a plurality of sensor signals. In some embodiments, a plurality of different trained models may be evaluated to determine a best estimator for cuffless and continuous measurement of a BP value, such as, for example, systolic pressure, diastolic pressure and mean arterial pressure (MAP).

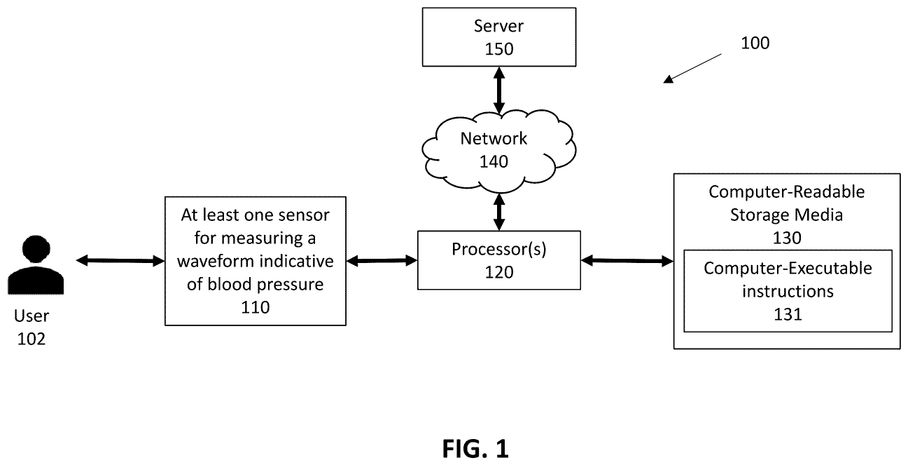

FIG. 1 illustrates a block diagram of the system 100 with which some embodiments may operate. In some embodiments, the system 100 comprises at least one sensor 110. The system 100, when engaged with at least one user 102, can measure one or more waveforms from the at least one sensor 110, such as, for example, a photoplethysmography (PPG) sensor, an electrocardiogram (ECG) sensor, or a combination thereof, for a plurality of features for priority and designation for different BP measurements from the user 102. In some embodiments, the system 100 can include a processor(s) 120, which may be combined with the at least one sensor 110 on a computing device or other suitable device, for operating the at least one sensor 110. In some embodiments, the system 100 can also include computer-readable storage media 130, which may be combined with the at least one sensor 110 and/or the processor(s) 120 on the computing device or other suitable device. The computer-readable storage media 130 may comprise computer-executable instructions 131 that, when executed by the processor(s), cause the processor(s) 120 to carry out methods for estimating continuous BP, described in more detail herein.

In some embodiments, the processor(s) 120 may be configured at least to operate the at least one sensor 110, to carry out methods for estimating continuous BP, or any combination thereof. In some embodiments, the processor(s) 120 may be configured to relay data, i.e., the one or more waveforms from the at least one sensor 110, from at least one sensor 110 to computer-readable storage media 130 for storage and/or analysis.

In some embodiments, the processor(s) 110 may be a remote control (RC) device. In some embodiments, the at least one sensor 110 may be controlled remotely by the RC device. In some embodiments, the RC device is wireless or wired. In some embodiments, the wireless RC device may be a mobile phone, dedicated wireless remote controller, or other control device. In some embodiments, the RC device comprises applications and cloud service. The term “cloud service” refers to a service that is provided over a network connection, via a non-local computer. Alternate types of RCs could be used in place of a phone, including, for example, an electronic tablet or a laptop or desktop computer.

In some embodiments, at least one of the at least one sensor 110, the processor(s) 120, the computer-readable storage media 130, or combinations thereof are housed together within the computing device or other suitable device. In some embodiments, the at least one sensor 110, the processor(s) 120, and the computer-readable storage media 130 are in separate devices.

In some embodiments, the at least one sensor 110 comprises a microprocessor for independently operating the at least one sensor 110 independent from the processor(s) 120. In some embodiments, the microprocessor is wirelessly connected to the processor(s) 120. In some embodiments, the microprocessor may relay the one or more waveforms to any of the processor(s) 120 and/or the computer-readable storage media 130.

In some embodiments, one or more of the at least one sensor 110 (which may include a microprocessor), the processor(s) 120, the computer-readable storage media 130, or any combination thereof is wearable. The term “wearable” herein means wearable around a finger, limb, body surface, head, etc., and/or may be incorporated into an arm band, wrist band, leg band, ankle band, bracelet, finger ring, or watch strap. The term “wearable” may also mean wearable in a single wearable unit in any combination or in individual wearable units. In some embodiments, only the at least one sensor 110 is wearable. In some embodiments, the at least one sensor 110 and the processor(s) 120 are wearable. In some embodiments, the at least one sensor 110, the processor(s) 120 and the computer-readable storage media 130 are wearable. In some embodiments, only the at least one sensor 110 and a microprocessor of the at least one sensor 110 is wearable.

In some embodiments, the system 100 can also include a network 140 and/or a server 150. In some embodiments, the network 140 may be configured to facilitate communications among the processor(s) 120 and the external server 150. The network 140 can be or include any one or more wired and/or wireless, local- and/or wide-area networks, including one or more enterprise networks and/or the Internet. In some embodiments, data, i.e., the one or more waveforms from the one or more sensor, may be transmitted by the processor(s) 120 to the server 150 via the network 140 for storage and/or analysis. In some embodiments, the data may be transmitted by the microprocessor of the at least one sensor 110 to the server 150 via the network 140 for storage and/or analysis. In some embodiments, the network 140 comprises Bluetooth®, Bluetooth® low energy, mobile, Wi-Fi or other communications protocols and modalities. The term “Bluetooth Low Energy (BLE)” refers to a wireless communication protocol that is similar to, but independent of, traditional Bluetooth, that permits devices to communicate over short distances with lower energy. Although BLE is independent of Bluetooth, the two protocols can be supported by a single device and can use a single antenna.

In some embodiments, one or more of the at least one sensor 110, the processor(s) 120, the computer-readable storage media 130, or any combination thereof of the system 100 may further include a user interface by which the user 102 may interact with the at least one sensor 110, the processor(s) 120, and/or the computer-readable storage media 130. For example, the user 102 can use the user interface to interact, collectively or individually, with the computer-readable storage media 130, network 140, or server 150. For example, the user 102 may operate the user interface to initiate analysis of the one or more waveforms and display analysis results such as whether there is an optimal trained model or combination thereof for any BP measurements the interface. The user 102 may additionally or alternatively operate the user interface to operate the at least one sensor or input the one or more waveforms. Those waveforms may be analyzed by the processor(s) 120 or a microprocessor of the at least one sensor 110. As a further example, the user 102 may operate the user interface to initiate analysis of the one or more waveforms by the computer-executable instruction 131 and provision of analysis results (e.g., hyperparameters, mean error, absolute error, etc.) from the computer-executable instruction 131 via the network 140. Results of analysis of the results (received from the computer-executable instruction 131 or from the interface) by the processor 120 may be output to the user interface, such as by being received and/or displayed at the user interface. In some embodiments, as mentioned above, the user interface may include a web interface, such as one or more web pages into which values may be output and which may display results of the analysis by the process 120, but embodiments are not so limited. The user interface may accept input in a variety of different formats, such as through speech recognition, text input, or other means, as embodiments are not limited in this respect.

In some aspects, the techniques described herein relate to a method for estimating blood pressure, the method including: (a) receiving a feature set from a user, the feature set including a plurality of measured temporal features and a plurality of measured morphology features measured by a photoplethysmphographic (PPG) and/or electrocardiogram (ECG) sensor, and, optionally, a set of biometric features; (b) applying the feature set to one or more trained models, which are trained for each of a systolic blood pressure, a diastolic blood pressure, and/or a mean arterial pressure; (c) determining an optimal trained model or combination thereof out of one or more trained models; and (d) extracting a user systolic blood pressure, a user diastolic blood pressure, and/or a user mean arterial pressure from the optimal trained model or combination thereof out of one or more trained models.

In some aspects, each of the systolic BP and diastolic BP may be estimated from a single trained model or from an ensemble of trained models. In some aspects, the plurality of temporal features include one or more of: pulse rate, a pulse arrival time, and a pulse transit time. In some aspects, the pulse arrival time is measured by a time difference between an ECG R-wave peak and the ensuing valley peak from a PPG waveform. In some aspects, the pulse transit time is measured by a time difference between subsequent peaks of two PPG waveforms separated spatially. In some aspects, the plurality of morphology features include one or more of: blood pressure cycle time, ejection or artery fill time, artery emptying time, peak volume, systolic volume, systolic volume differential (including a double differential), diastolic volume, and diastolic volume differential (including a double differential). In some aspects, each of the one or more ML algorithms is trained using sample data, the sample data including a plurality of temporal feature samples, morphology feature samples and biometric feature samples. In some aspects, each of the plurality of temporal feature samples and the plurality of morphology feature samples are obtained from one or more of healthy subjects and/or hemodynamically compromised subjects, wherein the hemodynamically compromised subjects have abnormal blood pressure due to a physiological disease.

In some aspects, a technique for each of the plurality of models comprises a machine learning (ML) model selected from one or more of: a Lasso algorithm, a Random Forest (RF) algorithm, a Support Vector Machine (SVM) algorithm, an Artificial Neural Network (ANN) algorithm, and a Long Short Term Memory (LSTM) algorithm, or a Residual Network (RESNET) deep learning algorithm. In some aspects, the technique is selected based on a lowest mean absolute error and/or standard deviation of a model output from the sample data. In some aspects, the hyperparameter tuning for the Lasso algorithm includes an alpha parameter which is defined as the elastic net regularization parameter which is the weight between lasso (1) and ridge regression(0). For example, the alpha parameter may be set based on the lowest standard deviation obtained for a range of alpha values between 0.6 and 1.0 for systolic and diastolic pressure separately. In some aspects, the hyperparameter for the RF algorithm includes number of splits or nodes of the decision trees and a number of learning cycles. For example, the hyperparameter tuning for the number of splits and learning cycles may be determined by the lowest standard deviation obtained for a range of split values such as between 80 to 160 and learning cycles such as between 300 to 700 for systolic and diastolic pressure. . . . In some aspects, the hyperparameter tuning for the SVM algorithm consisted of the box constraint and a kernel scale and was determined by the lowest standard deviation obtained for a range of box constraint values, for example, between 0.001 to 166 and kernel scale values, for example, between 0.31 and 0.82 for diastolic and systolic pressure. In some aspects, the hyperparameter tuning for each of the ANN algorithm and LSTM algorithm includes an epoch value and may be determined by the lowest standard deviation obtained for a range of epoch values, for example, between 500 and 2500 for diastolic and systolic pressures separately. In some aspects, the first trained model for diastolic pressure is different from the second trained model for systolic pressure. In some aspects, each of the first trained model and the second trained model is trained using a leave one out methodology. In some aspects, a method of each of the first trained model and the second trained model is selected based on one or more of a bias error and a standard deviation error.

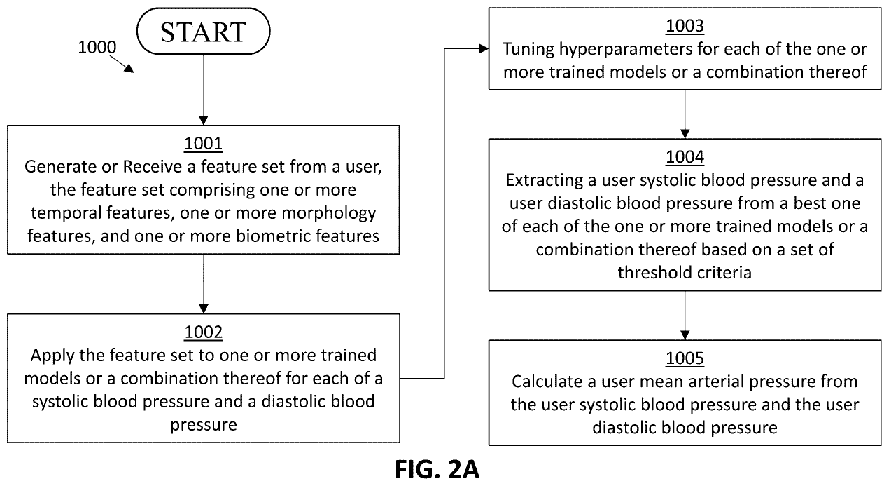

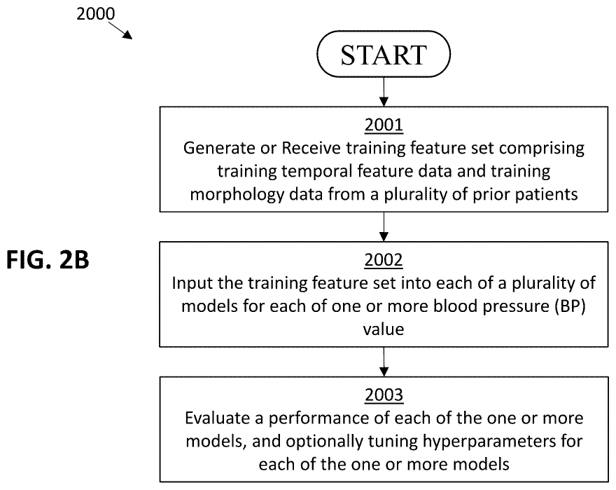

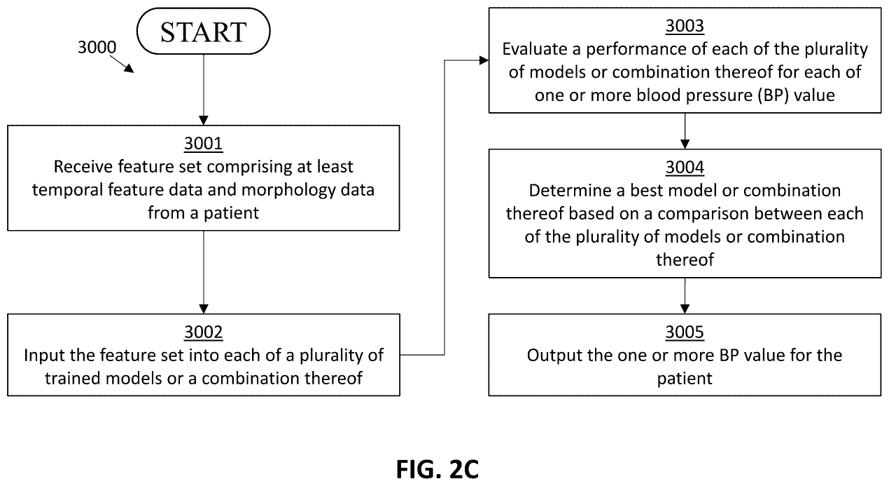

FIGS. 2A-2C are flowcharts illustrating example processes 1000, 2000 and 3000 used by a system 100 to interact with a user via an interactive interface, as depicted in FIG. 1. In regard to FIG. 2A, the process 1000 begins at a start step, and then moves to a step 1001, wherein the system 100 generates or receives a feature set from a user, the feature set comprising one or more temporal features, one or more morphology features and one or more biometric features. In some embodiments, the one or more morphology features comprise: a BP cycle time, an ejection time, an artery fill time, an artery emptying time, a peak volume, a systolic volume, a systolic volume differential, a diastolic volume, a diastolic volume differential, or a combination thereof. In some embodiments, the temporal or morphology features may be measured by one or more sensors, such as, for example, at least two photoplethysmography (PPG) sensors or an ECG and a photoplethysmography (PPG) sensor. This can occur when a user is operating a system 100 or opens a system application. The user can access the interactive interface 111 over the network 140 on an internet-enabled system 100. In some embodiments, the at least one of the one or more temporal features and the one or more morphology features are extracted from: a first PPG sensor and a second PPG sensor, a first PPG sensor and a first electrocardiogram (ECG) sensor, or a first ECG sensor and a second ECG sensor. In some embodiments, the one or more morphology features are obtained from at least one PPG waveform, wherein the at least one PPG waveform is collected noninvasively over a radial artery. In some embodiments, the one or more temporal features may comprise a pulse arrival time (PAT), a pulse transit time (PTT), a pulse rate, and/or a combination thereof. In some embodiments, the one or more biometric features comprises one or more of: a pre-existing condition, an age, a weight, a height, a waist size, a body mass index (BMI), a sex, and/or a combination thereof.

Next, the process moves to step 1002, wherein the system 100 applies the feature set to one or more trained models or a combination thereof for each of a systolic blood pressure and a diastolic blood pressure; and step 1003 where hyperparameter tuning is performed. The stored user information can be associated with the user's account. The trained models and features can be stored in the computer-readable storage media 130. In some embodiments, the one or more trained model comprises one or more of: a lasso model, a random forest model, a support vector machine model, an artificial neural network model, a long short term memory model, a RESNET deep learning model, and/or a combination or ensemble thereof. In some embodiments, the ensemble is a parallel or serial configuration of the one or more trained models. Next, the process moves to step 1004, wherein the system 100 extracts a user systolic blood pressure from and a user diastolic blood pressure from the optimum of the one or more trained models or a combination thereof. Next, the process moves to step 1005, wherein the system 100 calculates a user mean arterial pressure from the user systolic blood pressure and the user diastolic blood pressure. Further details for each of these steps are described herein.

In regard to FIG. 2B, the process 2000 may be performed for training each of one or more trained models, such that each of the one or more trained models are trained using at least a training feature set from a plurality of prior patients. Step 2001 comprises generating or receiving a training feature set comprising training temporal feature data and training morphology data from a plurality of prior patients. In some embodiments, each of the training temporal feature data and training morphology data may be obtained from external databases or internal databases. In some embodiments, each of the training temporal feature data and training morphology data may be obtained from each of one or more sensors, such as, for example, a first PPG sensor, a second PPG sensor, a first ECG sensor, a second ECG sensor, or a combination thereof. In some embodiments, each of the prior patients has a pre-existing condition. In some embodiments, the training feature set may further be preprocessed prior to proceeding to step 2002, as discussed in more detail below. In some embodiments, the preprocessing may comprise configuring the one or more features as a function of one or more of: a pre-ejection period, a square of pulse transit time, a photoplethysmography (PPG) intensity ratio, and/or a Womersly number. Step 2002 comprises inputting the training feature set into each of a plurality of models for each of one or more blood pressure (BP) value. In some embodiments, the BP value comprises one or more of a systolic BP, a diastolic BP, and/or a mean arterial pressure (MAP). Step 2003 comprises evaluating a performance of each of the plurality of models, and optionally tuning hyperparameters for each of the plurality of models in parallel. In some embodiments, step 2003 further comprises optionally further training and/or tuning one or each of the plurality of models to a specific patient or user.

In regard to FIG. 2C, the process 3000 may be performed by the user in estimating a BP values. Step 3001 comprises receiving a feature set comprising at least temporal feature data and morphology data from a patient. Step 3002 comprises inputting the feature set into each of a plurality of trained models or a combination thereof. Step 3003 comprises evaluating a performance of each of the plurality of models or combination thereof for each of one or more blood pressure (BP) value. Step 3004 comprises determining a best model or combination thereof based on a comparison between each of the plurality of models or combination thereof. In some embodiments, the determining comprises selecting the one or more trained models from a plurality of models, wherein the selecting is based on evaluating a performance of the one or more trained model as compared to others of the plurality of models. In some embodiments, the evaluating is based on one or more of an average error bias and/or a standard deviation of each of the plurality of models. In some embodiments, the evaluating is performed independently for each of the one or more BP value. In some embodiments, the determining comprises selecting an ensemble of two or more of the one or more trained models, wherein the selecting is based on evaluating a performance of the one or more trained model as compared to others of the plurality of models. In some embodiments, the ensemble comprises the two or more of the one or more trained models being in parallel or serial. Step 3005 comprises outputting the one or more BP value for the patient.

In some aspects, the techniques described herein relate to a computer-implemented method including: (a) generating a feature set from a user, the feature set including a plurality of measured temporal features, a plurality of measured morphology features and a plurality of biometric features; (b) applying the feature set to a combination of one or more trained models or a combination thereof, whose hyperparameters have been tuned for each of a systolic blood pressure and a diastolic blood pressure; (c) extracting a user systolic blood pressure and a user diastolic blood pressure from the optimum of each of the one or more trained models or a combination thereof; and (d) calculating a user mean arterial pressure from the user systolic blood pressure and the user diastolic blood pressure.

In some aspects, the at least one feature set may be subject to preprocessing. In some aspects, the preprocessing comprises configuring the inputs to the trained models as a combination of the following input features: Pulse arrival time (PAT), pulse rate, age, sex, BP cycle time (Tc), ejection or artery fill time (Ts, time in seconds from the start of the PPG waveform to the peak), artery emptying time (Td, time in seconds from the peak to the start of the next cycle), peak volume (Vp, volume depicted by the length from start of the PPG waveform to the peak), systolic volume (Vp times the ratio Ts/Tc), systolic volume differential (Vp divided by Ts), diastolic volume (Vp times Td/Tc) and diastolic volume differential (Vp divided by Td).

In some aspects, the preprocessing comprises configuring the inputs to the trained models as follows: the pulse arrival or transit time may be in a logarithmic form In (Pulse arrival or transit time). The pre-ejection period (PEP) may be in a logarithmic form In(PEP) and may be calculated as the difference between the Pulse arrival time (PAT) and Pulse Transit time(PTT). All the morphology features (Tc, Ts, Td, Vp, Vp times the ratio Ts/Tc, Vp divided by Ts, Vp times Td/Tc, Vp divided by Td) may be in logarithmic form In(morphology feature). In addition, two additional morphological features valley volume (Vv, depicted by the length from start of the PPG waveform when it begins to empty from the artery to the peak of the valley) and the peak to valley distance (Dpv, distance from peak to valley of the PPG wave) may be added to the input features in their logarithmic form. The heart rate may be in a logarithmic form In(heart rate).

In some aspects, the preprocessing comprises configuring the inputs to the trained models as a function of PEP, PTT, PIR and Womersley number as follows: pre-ejection period (PEP), square of pulse transit time (PTT²), PPG Intensity Ratio (PIR) approximated as the peak to valley distance (Dpv, distance from peak to valley of the PPG wave) and the Womersley number calculation approximated as Dpv (1-0.56/sqrt(2)*α) where α is the Womersley number approximated by Dpv, heart rate and Vv. This is based on the function: Pulse Pressure=f(PEP, PTT², PIR, Womersley number²).

In some aspects, the at least one feature set may be implemented independently through each of the five machine learning algorithms (Lasso, Random Forest, Support vector machine, Neural Network, LSTM and RESNET), and the algorithm giving the lowest standard deviation and bias combination may be chosen from the group as the most optimal algorithm.

In some aspects, the input features may be implemented through each of the five machine learning algorithms (Lasso, Random Forest, Support vector machine, Neural Network, LSTM and RESNET) as an ensemble and the algorithm giving the most optimal outcome at each node may be chosen from the ensemble, as the most optimal output for the subsequent node, thereby leading to the derivation of the optimal final output. An ensemble may be in parallel or serial.

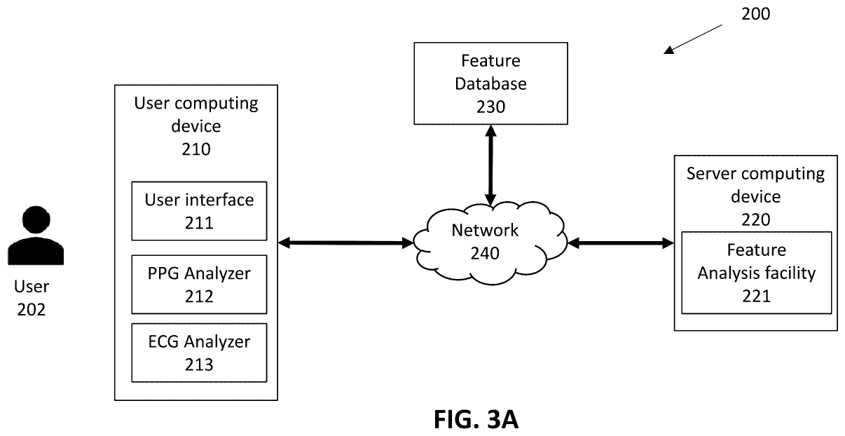

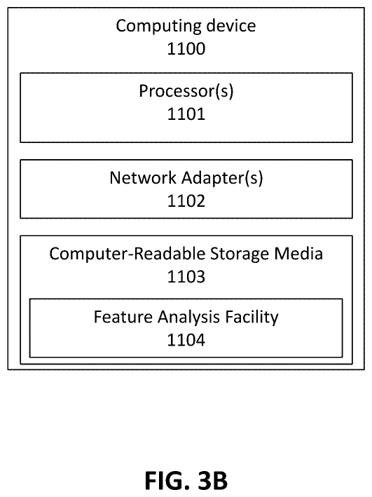

FIG. 3A illustrates a block diagram of an additional system 200 with which some embodiments may operate. The system 100 can analyze PPG signals, such as through a PPG analyzer 212, for a plurality of features for priority and designation for different BP measurements. The system 200 can include a user computing device 210, which may be a desktop or laptop personal computer, smart mobile phone, server, or other suitable device. The user computing device 210 may include a user interface 211 by which the user 202 may interact with the user computing device 210. For example, the user 202 can use the user interface 211 to interface with the feature database 230 or feature analysis facility 221 of the server computing device 220. For example, the user 202 may operate the user interface 211 to initiate analysis of a feature from the feature database 230 and display analysis results such as whether there is an optimal trained model or combination thereof for either the systolic, diastolic, or mean arterial pressures in the interface 211. The user 202 may additionally or alternatively operate the user interface 211 to input PPG waveforms using PPG analyzer 212 or ECG waveforms using ECG analyzer 213 obtained from the feature database 230, such as output to the user 202 in another interface. Those values may be provided to the feature analysis facility 221. As a further example, the user 202 may operate the user interface 211 to initiate analysis of the feature by the feature database 230 and provision of analysis results (e.g., hyperparameters, mean error, absolute error, etc.) from the feature database 230 to the feature analysis facility 221. Results of analysis of the results (received from the feature database 230 or from the interface 211) by the feature analysis facility 221 may be output to the user interface 211, such as by being received at the user interface 211 and displayed on the device 210. In some embodiments, as mentioned above, the user interface 211 may include a web interface, such as one or more web pages into which values may be output and which may display results of the analysis by the feature analysis facility 221, but embodiments are not so limited. The user interface 211 may accept input in a variety of different formats, such as through speech recognition, text input, or other means, as embodiments are not limited in this respect.

The system 200 can include a server computing device 220, which may include a feature analysis facility 221 configured to analyze factors (e.g., derived from the features, such as by the feature database 230) for the user 202 to rank or select trained models or combinations thereof. In some embodiments, the feature analysis facility 221 may receive information on the factors from the feature database 230 and/or from the user interface 211. In some embodiments, the feature analysis facility 221 may utilize the extracted systolic and diastolic blood pressure to satisfy predetermined error criteria for BP measurements.

The system 200 can include a network 240 to facilitate communications among the feature database 230, the user computing device 210, and the server computing device 220. The network 240 can be or include any one or more wired and/or wireless, local- and/or wide-area networks, including one or more enterprise networks and/or the Internet.

While the example of FIG. 3A includes the feature analysis facility 221 is illustrated on a different computing device from the user computing device 210 and the feature database 230, embodiments are not so limited. In some embodiments, the feature analysis facility may be implemented on the client computing device or the feature database 230. In some embodiments, the user interface 211 may not be separate from the feature analysis facility 221, but instead may be implemented as a single program or software application. In some embodiments, a feature database 230 may include the user interface 211 and the feature analysis facility 221, and the interface 211 and facility 216 may be implemented within the same program or application executed on the feature database 230.

In some embodiments, the temporal features may comprise one or more of pulse arrival time (PAT), pulse transit time (PTT), and pulse rate. In some embodiments, the morphology features comprise one or more of the following 8 features: BP cycle time (Tc), ejection or artery fill time (Ts, time in seconds from the start of the PPG waveform to the peak), artery emptying time (Td, time in seconds from the peak to the start of the next cycle), peak volume (Vp, volume depicted by the length from start of the PPG waveform to the peak), systolic volume (Vp times the ratio Ts/Tc), systolic volume differential (Vp divided by Ts), diastolic volume (Vp times Td/Tc) and diastolic volume differential (Vp divided by Td). In some embodiments, the biometric features comprise but are not limited to pre-existing condition, age, weight, height, waist size, BMI and/or sex.

The temporal features and the morphology features may be extracted from each of the plurality of ECG and/or PPG signals by identifying peak times for the ECG R-waves, PPG and the peaks and valleys for the PPG. PAT may be calculated by averaging the time difference between an ECG R-wave peak and the ensuing valley peak from a PPG waveform over a plurality of successive heart-beat waveforms, such as, for example, five waveforms, chosen via visual or automatic identification of artifact-free segments. The eight morphological features may be extracted from the plurality of successive PPG waveforms and averaged. Arterial systolic and diastolic pressures, measured using an intra-arterial catheter-based sensor inserted in the pulmonary artery, may be averaged over the plurality corresponding waveforms, forming the true systolic and diastolic values. A true MAP may be computed as follows:

$$MAP = \frac{( True Systolic BP ) + 2 \cdot ( True Diastolic BP )}{3} \qquad ( 1 )$$

In some embodiments, the plurality of different trained models comprise one or more of: i) Lasso—prediction based on a linear model using least squares regression coefficients on the feature data; ii) RF (Random Forest)—prediction based on a trained regression ensemble model that includes boosting a plurality of regression trees, such as, for example, 100; iii) SVM (Support Vector Machine)—prediction based on a support vector machine regression model using kernel functions; iv) ANN (Artificial Neural Network)—prediction using a two layer feed forward network containing a plurality of neurons, such as, for example, 20 neurons, in a hidden layer, a transfer function and a Levenberg-Marquardt backpropagation algorithm; v) LSTM (Long Short Term Memory)—prediction using an “ADAM” or adaptive moment estimation optimizer using a plurality of hidden neurons, such as, for example, 140 hidden neurons, a sequential input layer and a fully connected output layer; vi) RESNET deep learning algorithm—prediction using a multi-layer deep learning algorithm.

In some embodiments, each feature set may be used to train and independently test each of the plurality of trained models to produce a test dataset. In some embodiments, training and independently testing each of the plurality of trained models may comprise using a leave-one-out methodology where training may be performed on all but one datum, and the systolic pressure, diastolic pressure and MAP pressures may be estimated on a remaining datum point. This process may then be repeated such that all datum points can be left out over these repeated trainings and estimation and the mean absolute error averaged over all readings.

In some embodiments, hyperparameter selection may be evaluated for each of the plurality of trained models. For Lasso, the elastic net hyperparameter (“alpha”), which is an estimate of the Lasso to ridge variance, may be varied, for example, between 0.6 and 1.0. In RF, the two significant hyperparameters may comprise the number of decision tree splits and the number of learning cycles, which may be varied in five or more different combinations. For SVM, five or more optimal combinations of box constraint and kernel scale may be evaluated. Finally, for the neural network, LSTM and RESNET models, the number of epochs may be varied for a given constant learning rate. The hyperparameters producing the lowest standard deviation BP estimation error may then be chosen for each model for estimation of systolic pressure, diastolic pressure and MAP.

In some embodiments, each of the plurality of ML tests may be statistically compared to each other for each of three feature sets, comprising: the temporal features, the morphological features, and a combination of the temporal features and morphological features. For each of the plurality of trained models applied to user data, a bias (mean error) and standard deviation error between estimated systolic pressure, diastolic pressure and MAP may be measured and compared to labeled data, as well as their 95% confidence intervals. This may be repeated for the three feature sets. Two-way analysis of variance (ANOVA) may be used to test for significant differences in mean error between the plurality of ML methods and three feature sets. In some embodiments, when significant differences are found, post hoc pairwise F-tests may then be performed.

In some embodiments, Levene's tests may be conducted on each of a plurality of test datasets to statistically compare a mean absolute error (which is also a measure of standard deviation) between the plurality of ML methods and the three feature datasets. If significant differences are found without interaction, post hoc pairwise t-tests may be performed. If interactions are found, a user may perform post hoc pairwise t-test comparisons of all factor combinations based on the number of combinations. All post hoc analyses may use Bonferroni-Holm correction for multiple comparisons.

### Data Feature Extraction Module

Provided herein are methods for extracting continuous, cuffless BP estimation using PAT or PTT using a data feature extraction module (DFEM).

In some aspects, the techniques described herein relate to a system for estimating blood pressure, the system including: a data feature extraction module, the data feature extraction module including a plurality of ECG and/or PPG sensors, and a blood pressure estimation module, the blood pressure estimation module configured to carry out a method including: (a) generating a feature set from a user, the feature set including a plurality of measured temporal features and a plurality of measured morphology features from the plurality of ECG and/or PPG sensors, and biometric features; (b) applying the feature set to a combination of one or more machine learning (ML) algorithms for each of a systolic blood pressure and a diastolic blood pressure, and evaluating a hyperparameter selection for each of the one or more trained models; (c) extracting a user systolic blood pressure and a user diastolic blood pressure from each of the one or more trained models; and (d) calculating a user mean arterial pressure from the user systolic blood pressure and the user diastolic blood pressure.

In some aspects, the plurality of PPG sensors includes one or more of an infrared LED and detector for measuring a PPG. In some aspects, the plurality of PPG sensors includes two PPG sensor LEDs separated by a minimum of 5 mm along with a detector LED. In some aspects, the system is wearable. In some aspects, the plurality of temporal features include one or more of: pulse rate, a pulse arrival time, and a pulse transit time. In some aspects, the plurality of temporal features includes two ECG electrodes along with two PPG sensor LEDs separated by a minimum of 5 mm along with a detector LED.

In some aspects, the techniques described herein relate to an apparatus including: at least one processor; and at least one computer-readable storage medium having encoded thereon executable instructions that, when executed by the at least one processor, cause the at least one processor to carry out a method provided herein.

The PAT, for example, may be measured using the ECG R-wave and the pulse valley peak at the distal end. A time delay between the ECG R-wave to the arrival of the pulse peak at a distal location such as the wrist is the PAT, while a time delay between the pulse peak at a proximal location to its arrival at a distal location such as the wrist is defined as the PTT. If PAT is measured using a location just distal to the heart, it includes the pre-ejection period (PEP)—the time associated with ejection of blood from the heart into the aorta. PAT and PTT are inversely related to pulse wave velocity if the distance between the proximal and distal points of measurement is maintained constant. PAT or PTT have become well-established physiological correlates of systolic pressure, diastolic pressure, and MAP.

In some embodiments, the DFEM comprises at least two sensors. In some embodiments, the at least two sensors comprise photoplethysmography (PPG) sensors. In some embodiments, the at least two PPG sensors comprise LEDs. In some embodiments, each PPG sensor may be separated by a distance, for example, 5 mm, along with a detector that is used to capture a plurality of, for example, two, PPG waveforms (separated in space) along with their time stamps. In some embodiments, the emitter comprises an LED and the detector comprises a photodetector. A time difference between subsequent peaks of at least two PPG waveforms is the pulse transit time (PTT) and is used as a temporal feature along with the pulse rate and the biometric data. Each of the plurality of PPG waveforms may be annotated for a waveform morphology feature as discussed above. In some embodiments, each of the plurality of PPG waveforms may be red and infrared PPG waveforms. In some embodiments, each of the plurality of PPG waveforms may be sampled at a particular rate, for example, 400 Hz, at a particular resolution, for example, 19 bits resolution. In some embodiments, each of the plurality of PPG waveforms may be simultaneously collected noninvasively over a radial artery on a left or right wrist of a user using a watch with the aforementioned sensors. In some embodiments, a distal sensor pair may be located over a radial artery, such as, for example, approximately 2 cm from a wrist crease. For BP estimation, PTT (over the a particular distance, for example, 5 mm), pulse rate, and a plurality of biometric factors (e.g., height, waist size, weight, sex, body mass index (BMI), age) may be extracted for additional input features. In some embodiments, the weight and temperature can be measured using commercially available weight scales and BMI measurement devices.

In some embodiments, the DFEM is based on a two ECG sensor electrodes and an ECG waveform app. In some embodiments, two commecially available ECG electrodes one for finger of each hand and the ECG waveform measurement app. In some embodiments, the ECG waveform is integrated with the PPG waveforms on the time scale to measure the time difference between the peak of the ECG-R wave and peak of the PPG waveform to estimate the pulse arrival time and using the pulse transit time between the peaks of the two PPG sensor waveforms to estimate the pre-ejection period. A seismocardiogram using an optical sensor array, a ballistocardiogram using integrated strain gages, a reflective optical sensor array, Hall effect sensors, bioimpedance using an impedance electrode array or pressure tonography waveforms may also be used as substitutes for the PPG signal.

### Computer-Implementation

Techniques operating according to the principles described herein may be implemented in any suitable manner. Included in the discussion above are a series of flow charts showing the steps and acts of various processes that estimate blood pressure values for a patient. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application, for example as a software program application such as a signal analysis facility.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 1103 of FIG. 3B described below (i.e., as a portion of a computing device 1100) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 3A, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing devices (co-located or geographically distributed) dedicated to executing the techniques described herein, one or more Field-Programmable Gate Arrays (FPGAs) for carrying out the techniques described herein, or any other suitable system.

FIG. 3B illustrates one exemplary implementation of a computing device in the form of a computing device 1100 that may be used in a system implementing techniques described herein, although others are possible. It should be appreciated that FIG. 3B is intended neither to be a depiction of necessary components for a computing device to execute a feature analysis facility 1104 in accordance with the principles described herein, nor a comprehensive depiction.

Computing device 1100 may comprise at least one processor 1101, a network adapter 1102, and computer-readable storage media 1103. Computing device 1100 may be, for example, a desktop or laptop personal computer, a personal digital assistant (PDA), a smart mobile phone, a server, a wireless access point or other networking element, or any other suitable computing device. Network adapter 1102 may be any suitable hardware and/or software to enable the computing device 1100 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 1103 may be adapted to store data to be processed and/or instructions to be executed by processor 1101. Processor 1101 enables processing of data and execution of instructions. The data and instructions may be stored on the computer-readable storage media 1103.

The data and instructions stored on computer-readable storage media 1103 may comprise computer-executable instructions implementing techniques which operate according to the principles described herein. In the example of FIG. 3B, computer-readable storage media 1103 stores computer-executable instructions implementing various facilities and storing various information as described above. Computer-readable storage media 1103 may store feature analysis facility 1104.

While not illustrated in FIG. 3B, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

### Additional Embodiments

In some aspects, the techniques described herein relate to a system for estimating blood pressure including: at least one sensor configured to measure at least one waveform related to blood pressure from a patient; at least one processor; and at least one computer-readable storage medium having encoded thereon executable instructions that, when executed by the at least one processor, cause the at least one processor to carry out a method including: (a) receiving, from the at least one sensor, the at least one waveform and determining, from the at least one waveform, one or more features including at least one or more temporal features and one or more morphology features; (b) analyzing the one or more features using one or more trained models to determine one or more blood pressure (BP) value associated with the patient; and (c) outputting the one or more BP value determined for the patient based on the received one or more features. In some aspects, the techniques described herein relate to a system, further including a user interface. In some aspects, the techniques described herein relate to a system, wherein at least one of the at least one sensor, the at least one processor, and the at least one computer-readable storage medium is wearable. In some aspects, the techniques described herein relate to a system, wherein the at least one sensor includes a photoplethysmography (PPG) sensor or an electrocardiogram (ECG) sensor. In some aspects, the techniques described herein relate to a system, wherein each of the one or more BP value includes one or more of a systolic BP, a diastolic BP, and/or a mean arterial pressure (MAP). In some aspects, the techniques described herein relate to a system, wherein the received one or more features is a preprocessed feature set, the preprocessed feature set including the one or more features configured as a function of one or more of: a pre-ejection period, a square of pulse transit time, a PPG intensity ratio, and/or a Womersly number. In some aspects, the techniques described herein relate to a system, wherein the one or more trained models includes one or more of: a lasso model, a random forest model, a support vector machine model, an artificial neural network model, a long short term memory model, a RESNET deep learning model, and/or a combination or ensemble thereof. In some aspects, the techniques described herein relate to a system, wherein the analyzing includes tuning hyperparameters for each of the one or more trained models. In some aspects, the techniques described herein relate to a system, wherein the one or more morphology features include: a BP cycle time, an ejection time, an artery fill time, an artery emptying time, a peak volume, a systolic volume, a systolic volume differential, a diastolic volume, a diastolic volume differential, or a combination thereof. In some aspects, the techniques described herein relate to a system, wherein the one or more morphology features are obtained from at least one PPG waveform, wherein the at least one PPG waveform is collected noninvasively over a radial artery. In some aspects, the techniques described herein relate to a system, wherein the one or more features further includes biometric data, wherein the biometric data includes one or more of: a pre-existing condition, an age, a weight, a height, a waist size, a body mass index (BMI), a sex, and/or a combination thereof. In some aspects, the techniques described herein relate to a system, wherein the one or more temporal features includes one or more of: a pulse arrival time (PAT), a pulse transit time (PTT), a pulse rate, and/or a combination thereof. In some aspects, the techniques described herein relate to a system, wherein the PAT is based on a time difference between a peak of an ECG-R wave and a peak of a PPG waveform. In some aspects, the techniques described herein relate to a system, wherein the PTT is based on a difference between at least two PPG waveforms. In some aspects, the techniques described herein relate to a system, wherein at least one of the one or more temporal features and the one or more morphology features are extracted from: a first PPG sensor and a second PPG sensor, a first PPG sensor and a first ECG sensor, or a first ECG sensor and a second ECG sensor. In some aspects, the techniques described herein relate to a system, wherein each of the one or more trained models are trained using at least training temporal feature data and training morphology data from a plurality of prior patients. In some aspects, the techniques described herein relate to a system, wherein each of the prior patients has a pre-existing condition. In some aspects, the techniques described herein relate to a system, wherein the determining includes selecting the one or more trained models from a plurality of models, wherein the selecting is based on evaluating a performance of the one or more trained models as compared to others of the plurality of models. In some aspects, the techniques described herein relate to a system, wherein the evaluating is based on one or more of an average error bias and/or a standard deviation of each of the plurality of models. In some aspects, the techniques described herein relate to a system, wherein the evaluating is performed independently for each of the one or more BP value. In some aspects, the techniques described herein relate to a system, wherein the determining includes selecting an ensemble of two or more of the one or more trained models, wherein the selecting is based on evaluating a performance of the one or more trained models as compared to others of the one or more trained models. In some aspects, the techniques described herein relate to a system, wherein the ensemble includes the two or more of the one or more trained models being in parallel or serial.

In some aspects, the techniques described herein relate to a method for estimating blood pressure, the method including: (a) determining, based on a received feature set of one or more features from a patient, one or more blood pressure (BP) value associated with the patient, the one or more features including at least one or more temporal features and one or more morphology features, the determining including analyzing the one or more features using one or more trained models, wherein each of the one or more trained models are trained using at least training temporal feature data and training morphology data from a plurality of prior patients; and (b) outputting the one or more BP value determined for the patient based on the received feature set. In some aspects, the techniques described herein relate to a method, wherein the one or more BP value includes one or more of a systolic BP, a diastolic BP, and/or a mean arterial pressure (MAP). In some aspects, the techniques described herein relate to a method, wherein the determining further includes preprocessing the received feature set prior to the analyzing, the preprocessing including configuring the one or more features as a function of one or more of: a pre-ejection period, a square of pulse transit time, a photoplethysmography (PPG) intensity ratio, and/or a Womersly number. In some aspects, the techniques described herein relate to a method, wherein the one or more trained models includes one or more of: a lasso model, a random forest model, a support vector machine model, an artificial neural network model, a long short term memory model, a RESNET deep learning model, and/or a combination or ensemble thereof. In some aspects, the techniques described herein relate to a method, wherein the analyzing includes tuning hyperparameters for each of the one or more trained models. In some aspects, the techniques described herein relate to a method, wherein the one or more morphology features include: a BP cycle time, an ejection time, an artery fill time, an artery emptying time, a peak volume, a systolic volume, a systolic volume differential, a diastolic volume, a diastolic volume differential, or a combination thereof. In some aspects, the techniques described herein relate to a method, wherein the one or more morphology features are obtained from at least one PPG waveform, wherein the at least one PPG waveform is collected noninvasively over a radial artery. In some aspects, the techniques described herein relate to a method, wherein the feature set further includes biometric data, wherein the biometric data includes one or more of: a pre-existing condition, an age, a weight, a height, a waist size, a body mass index (BMI), a sex, and/or a combination thereof. In some aspects, the techniques described herein relate to a method, wherein the one or more temporal features includes one or more of: a pulse arrival time (PAT), a pulse transit time (PTT), a pulse rate, and/or a combination thereof. In some aspects, the techniques described herein relate to a method, wherein the PAT is based on a time difference between a peak of an ECG-R wave and a peak of a PPG waveform. In some aspects, the techniques described herein relate to a method, wherein the PTT is based on a difference between at least two PPG waveforms. In some aspects, the techniques described herein relate to a method, wherein at least one of the one or more temporal features and the one or more morphology features are extracted from: a first PPG sensor and a second PPG sensor, a first PPG sensor and a first electrocardiogram (ECG) sensor, or a first ECG sensor and a second ECG sensor. In some aspects, the techniques described herein relate to a method, wherein each of the plurality of prior patients has a pre-existing condition. In some aspects, the techniques described herein relate to a method, wherein the determining includes selecting the one or more trained models from a plurality of models, wherein the selecting is based on evaluating a performance of the one or more trained models as compared to others of the plurality of models. In some aspects, the techniques described herein relate to a method, wherein the evaluating is based on one or more of an average error bias and/or a standard deviation of each of the plurality of models. In some aspects, the techniques described herein relate to a method, wherein the evaluating is performed independently for each of the one or more BP value. In some aspects, the techniques described herein relate to a method, wherein the determining includes selecting an ensemble of two or more of the one or more trained models, wherein the selecting is based on evaluating a performance of the one or more trained models as compared to others of the plurality of models. In some aspects, the techniques described herein relate to a method, wherein the ensemble includes the two or more of the one or more trained models being in parallel or serial.

In some aspects, the techniques described herein relate to at least one storage medium having encoded thereon executable instructions that, when executed by at least one processor, cause the at least one processor to carry out a method including: (a) determining, based on a received feature set of one or more features from a patient, one or more blood pressure (BP) value associated with the patient, the one or more features including at least one or more temporal features and one or more morphology features, the determining including analyzing the one or more features using one or more trained models, wherein each of the one or more trained models are trained using at least training temporal feature data and training morphology data from a plurality of prior patients; and (b) outputting the one or more BP value determined for the patient based on the received feature set. In some aspects, the techniques described herein relate to an at least one storage medium, wherein the one or more BP value includes one or more of a systolic BP, a diastolic BP, and/or a mean arterial pressure (MAP). In some aspects, the techniques described herein relate to an at least one storage medium, wherein the determining further includes preprocessing the received feature set prior to the analyzing, the preprocessing including configuring the one or more features as a function of one or more of: a pre-ejection period, a square of pulse transit time, a photoplethysmography (PPG) intensity ratio, and/or a Womersly number. In some aspects, the techniques described herein relate to an at least one storage medium, wherein the one or more trained models includes one or more of: a lasso model, a random forest model, a support vector machine model, an artificial neural network model, a long short term memory model, a RESNET deep learning model, and/or a combination or ensemble thereof. In some aspects, the techniques described herein relate to an at least one storage medium, wherein the analyzing includes tuning hyperparameters for each of the one or more trained models. In some aspects, the techniques described herein relate to an at least one storage medium, wherein the one or more morphology features include: a BP cycle time, an ejection time, an artery fill time, an artery emptying time, a peak volume, a systolic volume, a systolic volume differential, a diastolic volume, a diastolic volume differential, or a combination thereof. In some aspects, the techniques described herein relate to an at least one storage medium, wherein the one or more morphology features are obtained from at least one PPG waveform, wherein the at least one PPG waveform is collected noninvasively over a radial artery. In some aspects, the techniques described herein relate to an at least one storage medium, wherein the feature set further includes biometric data, wherein the biometric data includes one or more of: a pre-existing condition, an age, a weight, a height, a waist size, a body mass index (BMI), a sex, and/or a combination thereof. In some aspects, the techniques described herein relate to an at least one storage medium, wherein the one or more temporal features includes one or more of: a pulse arrival time (PAT), a pulse transit time (PTT), a pulse rate, and/or a combination thereof. In some aspects, the techniques described herein relate to an at least one storage medium, wherein the PAT is based on a time difference between a peak of an ECG-R wave and a peak of a PPG waveform. In some aspects, the techniques described herein relate to an at least one storage medium, wherein the PTT is based on a difference between at least two PPG waveforms. In some aspects, the techniques described herein relate to an at least one storage medium, wherein at least one of the one or more temporal features and the one or more morphology features are extracted from: a first PPG sensor and a second PPG sensor, a first PPG sensor and a first electrocardiogram (ECG) sensor, or a first ECG sensor and a second ECG sensor. In some aspects, the techniques described herein relate to an at least one storage medium, wherein each of the plurality of prior patients has a pre-existing condition. In some aspects, the techniques described herein relate to an at least one storage medium, wherein the determining includes selecting the one or more trained models from a plurality of models, wherein the selecting is based on evaluating a performance of the one or more trained models as compared to others of the plurality of models. In some aspects, the techniques described herein relate to an at least one storage medium, wherein the evaluating is based on one or more of an average error bias and/or a standard deviation of each of the plurality of models. In some aspects, the techniques described herein relate to an at least one storage medium, wherein the evaluating is performed independently for each of the one or more BP value. In some aspects, the techniques described herein relate to an at least one storage medium, wherein the determining includes selecting an ensemble of two or more of the one or more trained models, wherein the selecting is based on evaluating a performance of the one or more trained models as compared to others of the plurality of models. In some aspects, the techniques described herein relate to an at least one storage medium, wherein the ensemble includes the two or more of the one or more trained models being in parallel or serial.

In some aspects, the techniques described herein relate to a system for estimating blood pressure including: at least one processor; and at least one computer-readable storage medium having encoded thereon executable instructions that, when executed by the at least one processor, cause the at least one processor to carry out a method including: determining, based on a received feature set of one or more features from a patient, one or more blood pressure (BP) value associated with the patient, the one or more features including at least one or more temporal features and one or more morphology features, the determining including analyzing the one or more features using one or more trained models, wherein each of the one or more trained models are trained using at least training temporal feature data and training morphology data from a plurality of prior patients. In some aspects, the techniques described herein relate to a system, further including a user interface. In some aspects, the techniques described herein relate to a system, further including at least one sensor. In some aspects, the techniques described herein relate to a system, wherein at least one of the at least one sensor, the at least one processor, and the at least one computer-readable storage medium is wearable. In some aspects, the techniques described herein relate to a system, wherein the at least one sensor includes a photoplethysmography (PPG) sensor or an electrocardiogram (ECG) sensor. In some aspects, the techniques described herein relate to a system, wherein the one or more BP value includes one or more of a systolic BP, a diastolic BP, and/or a mean arterial pressure (MAP). In some aspects, the techniques described herein relate to a system, wherein the received one or more features is a preprocessed feature set, the preprocessed feature set including the one or more features configured as a function of one or more of: a pre-ejection period, a square of pulse transit time, a PPG intensity ratio, and/or a Womersly number. In some aspects, the techniques described herein relate to a system, wherein the one or more trained models includes one or more of: a lasso model, a random forest model, a support vector machine model, an artificial neural network model, a long short term memory model, a RESNET deep learning model, and/or a combination or ensemble thereof. In some aspects, the techniques described herein relate to a system, wherein the analyzing includes tuning hyperparameters for each of the one or more trained models. In some aspects, the techniques described herein relate to a system, wherein the one or more morphology features include: a BP cycle time, an ejection time, an artery fill time, an artery emptying time, a peak volume, a systolic volume, a systolic volume differential, a diastolic volume, a diastolic volume differential, or a combination thereof. In some aspects, the techniques described herein relate to a system, wherein the one or more morphology features are obtained from at least one PPG waveform, wherein the at least one PPG waveform is collected noninvasively over a radial artery. In some aspects, the techniques described herein relate to a system, wherein the one or more features further includes biometric data, wherein the biometric data includes one or more of: a pre-existing condition, an age, a weight, a height, a waist size, a body mass index (BMI), a sex, and/or a combination thereof. In some aspects, the techniques described herein relate to a system, wherein the one or more temporal features includes one or more of: a pulse arrival time (PAT), a pulse transit time (PTT), a pulse rate, and/or a combination thereof. In some aspects, the techniques described herein relate to a system, wherein the PAT is based on a time difference between a peak of an ECG-R wave and a peak of a PPG waveform. In some aspects, the techniques described herein relate to a system, wherein the PTT is based on a difference between at least two PPG waveforms. In some aspects, the techniques described herein relate to a system, wherein at least one of the one or more temporal features and the one or more morphology features are extracted from: a first PPG sensor and a second PPG sensor, a first PPG sensor and a first ECG sensor, or a first ECG sensor and a second ECG sensor. In some aspects, the techniques described herein relate to a system, wherein each of the one or more trained models are trained using at least training temporal feature data and training morphology data from a plurality of prior patients. In some aspects, the techniques described herein relate to a system, wherein each of the plurality of prior patients has a pre-existing condition. In some aspects, the techniques described herein relate to a system, wherein the determining includes selecting the one or more trained models from a plurality of models, wherein the selecting is based on evaluating a performance of the one or more trained models as compared to others of the plurality of models. In some aspects, the techniques described herein relate to a system, wherein the evaluating is based on one or more of an average error bias and/or a standard deviation of each of the plurality of models. In some aspects, the techniques described herein relate to a system, wherein the evaluating is performed independently for each of the one or more BP value. In some aspects, the techniques described herein relate to a system, wherein the determining includes selecting an ensemble of two or more of the one or more trained models, wherein the selecting is based on evaluating a performance of the one or more trained models as compared to others of the plurality of models. In some aspects, the techniques described herein relate to a system, wherein the ensemble includes the two or more of the one or more trained models being in parallel or serial.

In some aspects, the techniques described herein relate to a method for estimating blood pressure, the method including: (a) receiving at least one feature set obtained at distinct points in time after meeting certain acceptance criteria and averaged over a certain segment of data waveform from a user, the feature set including a plurality of measured temporal features and a plurality of measured morphology features measured by at least two photoplethysmography (PPG) sensors at either of a wrist, finger or ear canal locations, a pair of ECG sensors at two separate locations such as the sternum and one digit of the left or right hand or between two digits each of the right and left hand and a plurality of biometric characteristics such as but not restricted to age, height, weight, sex, waist size, Body Mass Index (BMI), pre-existing disease conditions; (b) applying the at least one feature set to a combination of one or more machine learning (ML) algorithms, whose hyperparameter selections have been tuned, for each of systolic blood pressure and diastolic blood pressure; (c) extracting a user systolic blood pressure and a user diastolic blood pressure from each of the one or more trained models and selecting a most optimal trained model or combination thereof for each; and (d) calculating a user mean arterial pressure from the user systolic blood pressure and the user diastolic blood pressure using an algebraic or other signal processing or mathematical technique.

In some aspects, the techniques described herein relate to a computer-implemented method including: (a) generating at least one feature set obtained at distinct points in time after meeting certain acceptance criteria and averaged over a certain segment of data waveform from a user, the feature set including a plurality of measured temporal features, a plurality of measured morphology features, and a plurality of biometric characteristics; (b) applying the at least one feature set(s) to a combination of one or more trained models whose hyperparameter selections have been tuned, for each of systolic blood pressure and diastolic blood pressure; (c) extracting a user systolic blood pressure and a user diastolic blood pressure from each of the one or more trained models and selecting a most optimal trained model or combination thereof for each; and (d) calculating a user mean arterial pressure from the user systolic blood pressure and the user diastolic blood pressure using an algebraic or other signal processing or mathematical technique.

In some aspects, the techniques described herein relate to a system for estimating blood pressure, the system including: a data feature extraction module, the data feature extraction module including a plurality of PPG sensors at the finger, wrist or ear and ECG sensors at the sternum or the digits, and a blood pressure estimation module, the blood pressure estimation module configured to carry out a method including: (a) generating at least one feature set obtained at distinct points in time after meeting certain acceptance criteria and averaged over a certain segment of data waveform from a user, the at least one feature set including a plurality of measured temporal features, a plurality of measured morphology features, and a plurality of biometric characteristics; (b) applying the feature set to a combination of one or more trained models for each of a systolic blood pressure and a diastolic blood pressure; (c) extracting a user systolic blood pressure and a user diastolic blood pressure from each of the one or more trained models; and selecting a most optimal trained model or combination thereof; and (d) calculating a user mean arterial pressure from the user systolic blood pressure and the user diastolic blood pressure using an algebraic or other signal processing or mathematical technique.

In some aspects, the techniques described herein relate to an apparatus including: at least one processor; and at least one computer-readable storage medium having encoded thereon executable instructions that, when executed by the at least one processor, cause the at least one processor to carry out a method provided herein.

### Example Systems and Methods

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments described herein, or combinations of one or more of these embodiments or aspects described therein may be employed in practicing the present disclosure. It is intended that the following claims define the scope of the present disclosure and that methods and structures within the scope of these claims and their equivalents be covered thereby.

### EXAMPLE 1

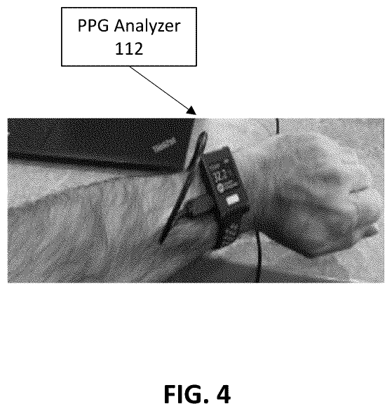

The purpose of this example is to provide a set of hyperparameters obtained from each ML method. FIG. 4 shows an exemplary experimental setup for data collection of the PPG waveform. TABLE 1 provides demographic information for healthy subjects as well as hemodynamically compromised subjects.

TABLE 1
Healthy and hemodynamically compromised subject demographics.

| | | MIMIC I hemodynamically |
|---|---|---|
| | Healthy subjects | compromised datasets |
| Descriptor | (n = 23) | (n = 126) |
| Male | 12 (52%) | 75 (60%) |
| Female | 11 (48%) | 51 (40%) |
| Age (>40 | 12 (52%) | 116 (92%) |
| years) | | |
| Age (<40 | 11 (48%) | 10 (8%) |
| years) | | |

TABLE 2 outlines the hyperparameters that may be evaluated for each method, based on input from the subjects in TABLE 1. For Lasso, the elastic net hyperparameter (“alpha”), which is an estimate of the Lasso to ridge variance, was varied between 0.6 and 1.0. In RF, the two significant hyperparameters consisted of the number of splits and the number of learning cycles, which were varied in five different combinations. For SVM, five optimal combinations of box constraint and kernel scale were evaluated. Finally, for the neural network and LSTM models, the number of epochs was varied for a given constant learning rate. The hyperparameters producing the lowest standard deviation BP estimation error (superscripts a and b in TABLE 2 below) were chosen for each model for estimation of systolic pressure and diastolic pressure, respectively.

TABLE 2
Hyperparameter selection.

| | | RF | | SVM | | | |
|---|---|---|---|---|---|---|---|
| | Lasso | | Learning | Box | Kernel | ANN | LSTM |
| Trial # | Alpha | Splits | Cycles | Constraint | Scale | Epochs | Epochs |
| 1 | 0.6 | 80^{a} | 300^{a} | 0.001 | 0.31 | 500 | 500 |
| 2 | 0.65 | 100 | 400 | 0.006 | 0.7 | 1000 | 1000^{a,b} |
| 3 | 0.7^{a} | 120^{b} | 500^{b} | 0.1 | 0.65 | 1500^{a} | 1500 |
| 4 | 0.75^{b} | 140 | 600 | 1 | 0.48 | 2000 | 2000 |
| 5 | 0.8 | 160 | 700 | 166^{a,b} | 0.82^{a,b} | 2500^{b} | 2500 |

^{a}Hyperparameters with the lowest systolic standard deviation
^{b}Hyperparameters with the lowest diastolic standard deviation.

### EXAMPLE 2

The purpose of this example is to provide exemplary BP data from datasets from hemodynamically comprised users from TABLE 1. TABLE 3 provides the mean, standard deviation and 95% confidence interval errors for the healthy dataset (n=23) using the leave-one-out method on all five ML methods and the pulse transit time feature dataset.

TABLE 3
Mean (μ), standard deviation (SD) and 95% confidence interval (CI) for signed errors (mmHg) in estimating mean
arterial pressure (MAP), systolic pressure and diastolic pressure for the hemodynamically compromised dataset.

| Lasso | | Random Forest | | SVM | | ANN | | LSTM | |
|---|---|---|---|---|---|---|---|---|---|
| μ ± SD | (95% CI) | μ ± SD | (95% CI) | μ ± SD | (95% CI) | μ ± SD | (95% CI) | μ ± SD | (95% CI) |

Healthy subjects (n = 23 subjects):

| | | | | | | | | | | |
|---|---|---|---|---|---|---|---|---|---|---|
| MAP | −0.18 ± | (−4.88, | 0.27 ± | (−3.62, | −2.18 ± | (−5.97, | −0.21 ± | (−4.36, | −10.15 ± | (−14.49, |
| | 11.51 | 4.53) | 9.51 | 4.16) | 9.26 | 1.60) | 10.15 | 3.94) | 10.61 | −5.82) |
| Systolic | 1.77 ± | (−7.41, | −0.65 ± | (−6.89, | 3.44 ± | (−2.10, | 1.27 ± | (−6.75, | 10.63 ± | (2.71, |
| | 22.46 | 10.95) | 15.28 | 5.59) | 13.56 | 8.98) | 19.62 | 9.29) | 19.37 | 18.55) |
| Diastolic | −0.62 ± | (−3.74, | −0.08 ± | (−3.14, | 1.56 ± | (−1.96, | −0.31 ± | (−3.89, | 9.92 ± | (4.40, |
| | 7.64^{a} | 2.50) | 7.49^{a} | 2.98) | 8.62 | 5.08) | 8.76 | 3.27) | 13.51 | 15.44) |

Hemodynamically compromised patients (N = 126 records from 31 subjects) for combined feature dataset:

| | | | | | | | | | | |
|---|---|---|---|---|---|---|---|---|---|---|
| MAP | 0.03 ± | (−2.05, | 0.76 ± | (−0.78, | 0.75 ± | (−1.09, | 1.17 ± | (−0.77, | 2.25 ± | (−0.09, |
| | 11.89 | 2.11) | 8.84 | 2.3) | 10.56 | 2.59) | 11.12 | 3.11) | 13.39 | 4.59) |
| Systolic | −0.14 ± | (−3.34, | 1.38 ± | (−1.26, | 0.43 ± | (−2.66, | 1.65 ± | (−2.13, | 3.78 ± | (−0.03, |
| | 18.31 | 3.06) | 15.12 | 4.02) | 17.7 | 3.52 | 21.62 | 5.43) | 21.82 | 7.59) |
| Diastolic | 0.03 ± | (−1.53, | 0.45 ± | (−0.86, | 0.91 ± | (−0.37, | (−0.93 ± | (−2.54, | 1.49 ± | (−0.11, |
| | 8.97 | 1.60) | 7.53^{a} | 1.76) | 7.32^{a} | 2.19) | 9.23 | 0.68) | 9.17 | 3.09) |

Hemodynamically compromised patients for PAT dataset (N = 126 records from 31 subjects):

| | | | | | | | | | | |
|---|---|---|---|---|---|---|---|---|---|---|
| MAP | 0.00 ± | (−2.21, | −0.08 ± | (−1.23, | 0.95 ± | (−0.74, | (−0.26 | (−1.67, | 2.32 ± | (−0.03, |
| | 12.67 | 2.21) | 6.57^{a} | 1.07) | 9.72 | 2.65) | 8.07 | 1.15) | 13.46 | 4.67) |
| Systolic | 0.00 ± | (−3.63, | −0.52 ± | (−3.21, | 0.63 ± | (−2.24, | (−1.52 ± | (−4.68, | 4.13 ± | (0.28, |
| | 20.8 | 3.63) | 15.4 | 2.17) | 16.44 | 3.5) | 18.12 | 1.64) | 22.03 | 7.98) |
| Diastolic | 0.00 ± | (−1.58, | 0.14 ± | (−0.65, | 1.09 ± | (−0.08, | 0.37 ± | (−0.66, | 1.42 ± | (−0.18, |
| | 9.03 | 1.58) | 4.51^{a} | 0.93) | 6.69^{a} | 2.26) | 5.92^{a} | 1.40) | 9.18 | 3.02) |

Hemodynamically compromised patients for morphology dataset (N = 126 records from 31 subjects):

| | | | | | | | | | | |
|---|---|---|---|---|---|---|---|---|---|---|
| MAP | 0.09 ± | (−2.08, | −0.14 ± | (−1.63, | 0.89 ± | (−0.87, | (−0.94 ± | (−2.93, | 1.36 ± | (−0.93, |
| | 12.46 | 2.27) | 8.55 | 1.35) | 10.11 | 2.66) | 11.41 | 1.05) | 13.1 | 3.65) |
| Systolic | −0.11 ± | (−3.39, | 0.09 ± | (−2.97, | 0.57 ± | (−2.44, | 2.49 ± | (−1.74, | 4.00 ± | (0.16, |
| | 18.83 | 3.18) | 17.51 | 3.15) | 17.24 | 3.58) | 24.22 | 6.72) | 21.99 | 7.84) |
| Diastolic | −0.14 ± | (−1.68, | 0.05 ± | (−1.28, | 1.01 ± | (−0.27, | 0.65 ± | (−1.37, | 0.045 ± | (−1.47, |
| | 8.8 | 1.40) | 7.61^{a} | 1.38) | 7.35^{a} | 2.30) | 11.55 | 2.67) | 8.66 | 1.56) |

^{a}Within 5 mm for bias and 8 mm for standard deviation.

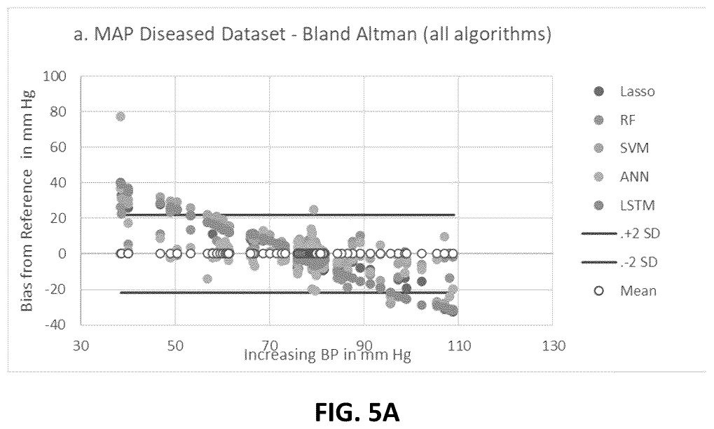

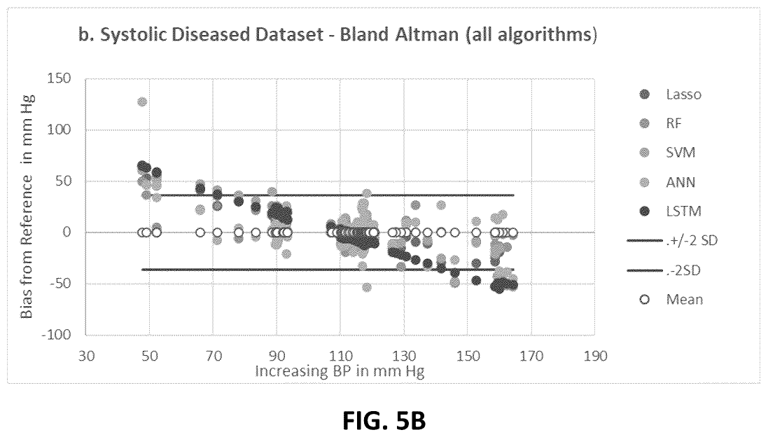

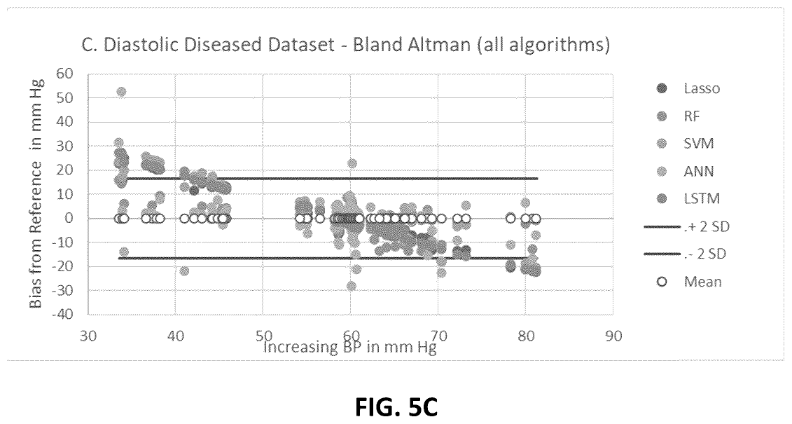

TABLE 3 also shows the mean, standard deviation and 95% confidence interval errors for the hemodynamically compromised subjects (n=31, 126 datasets) using the leave-one-out method on all five ML methods and all three feature datasets—combined, PAT and morphology. Every bias error met criterion 1 of the IEC 806-2-30:2018 standard, each being ≤5 mm Hg. Bland-Altman plots for the systolic pressure, diastolic pressure and MAP using the combined feature set and leave-one-out methodology and for each of the ML methods are shown in FIGS. 5A-C. In particular, FIGS. 5A-5C provide estimated BP in the independent test dataset using combined feature datasets from hemodynamically compromised patients (n=126). Lines show ±2 standard deviations of all five algorithms. FIG. 5A indicates MAP comparison, FIG. 5B provides systolic BP comparison, and FIG. 5C provides diastolic BP comparison. Axis scales differ between plots.

Two-way ANOVAs (one per pressure) were used to compare differences in means of the estimation errors between the five ML methods and three feature sets in the hemodynamically compromised dataset. Separately for each pressure, TABLE 4 shows that there was a significant difference between the five ML methods, but not the feature datasets, without interaction. Post hoc paired comparisons were conducted only on the combined feature dataset (since no significant differences were found between the three feature datasets and their data are highly correlated) between the five ML algorithms, as shown in TABLE 5. Only 11 of 30 pairwise comparisons between methods demonstrated a statistically significant difference. The LSTM method never showed statistically lower error performance. The SVM method most frequently showed statistically lower error performance (8 of the 11 differences).

TABLE 4
Two-way analysis of variance F-test and p-value results for significant differences in mean errors for mean, systolic
and diastolic BP using 5 ML methods and 3 feature sets in hemodynamically compromised subjects (N = 126).

| Method | Mean pressure | Systolic pressure | Diastolic pressure |
|---|---|---|---|
| Feature | F (2) = 0.04, p = 0.96 | F (2) = 0.06, p = 0.95 | F (2) = 0.065, p = 0.94 |
| Machine learning | F (4) = 4.88, p = 0.00065* | F (4) = 6.08, p = 0.000073* | F (4) = 3.58, p = 0.0065* |
| Interaction − Feature and ML | F (2, 4) = 0.05, p = 0.99 | F (2, 4) = 0.035, p = 0.99 | F (2, 4) = 0.11, p = 0.99 |

*significant difference

TABLE 5
Machine learning method - Post hoc statistical results
to test for differences in mean errors for each of the
machine learning methods using a combined feature dataset
in hemodynamically compromised subjects (N = 126).

| | |
|---|---|
| RF | SVM |

Mean

| | | |
|---|---|---|
| Lasso | NS | F (1) = 4.99, p = 0.007* (SVM) |
| RF | | NS |
| SVM | | |
| ANN | | |

Systolic

| | | |
|---|---|---|
| Lasso | NS | NS |
| RF | | F (1) = 5.36, p = 0.005* (RF) |
| SVM | | |
| ANN | | |

Diastolic

| Lasso | NS | F (1) = 5.6, p = 0.004* (SVM) |
|---|---|---|
| RF | | F (1) = 7.57, p = 0.001* (RF) |
| SVM | | |
| ANN | | |
| | ANN | LSTM |

Mean

| | | |
|---|---|---|
| Lasso | NS | NS |
| RF | NS | NS |
| SVM | NS | F (1) = 15.5, p = 0.000* (SVM) |
| ANN | | NS |

Systolic

| | | |
|---|---|---|
| Lasso | NS | F (1) = 7.02, p = 0.001* (Lasso) |
| RF | NS | F (1) = 11.9, p = 0.001* (RF) |
| SVM | NS | F (1) = 15.5, p = 0.000* (SVM) |
| ANN | | F (1) = 7.21, p = 0.007* (ANN) |

Diastolic

| | | |
|---|---|---|
| Lasso | NS | NS |
| RF | NS | NS |
| SVM | F (1) = 9.43, p = 0.002* (ANN) | F (1) = 9.05, p = 0.003*(SVM) |
| ANN | | NS |

*denotes a significant difference; NS indicates not significant.
Data are shown as F and p values.
Parentheses denote the method with the significantly lower mean error.

Separately for each pressure, the difference in absolute error (a measure of standard deviation) between the five ML methods and three feature sets was investigated using Levene's test, with results shown in TABLE 6. For diastolic BP, a significant difference was found between the five ML methods, but not among the feature datasets, without interaction. For MAP and systolic BP, the respective Levene's test each found an interaction (TABLE 6). Thus, for each pressure, post hoc pairwise comparisons were conducted between all combinations of the three feature datasets and five ML methods. TABLE 7 shows post hoc paired comparison results using the three feature datasets between the five ML algorithms.

TABLE 6
Levene's test results of significant differences in absolute error (standard deviation) for systolic pressure,
diastolic pressure and MAP in hemodynamically compromised subjects (N = 126 datasets from 31 subjects).

| Method | MAP | Systolic pressure | Diastolic pressure |
|---|---|---|---|
| Feature | F (2) = 2.50, p = 0.082 | F (2) = 2.59, p = 0.075 | F (2) = 2.47, p = 0.085 |
| Machine learning | F (4) = 22. 9, p = 0.00* | F (4) = 20.92, p = 0.00* | F (4) = 16.3, p = 0.00* |
| Interaction - Feature and ML | F (2, 4) = 2.54, p = 0.0095* | F (2, 4) = 2.81, p = 0.004* | F (2, 4) = 1.89, p = 0.058 |

*denotes a significant difference.
Data are shown as F and p values.

TABLE 7
Levene's test post hoc results to test for significant differences in absolute
error (standard deviation) for hemodynamically compromised subjects (N = 126
datasets from 31 subjects). For MAP, diastolic and systolic BP, the results are
shown for each pairwise combination of the feature dataset and machine learning method.

| | | | |
|---|---|---|---|
| RF | SVM | ANN | LSTM |

Mean

| | | | | |
|---|---|---|---|---|
| Lasso | p = 0.000 (RF) | NS | NS | NS |
| | [combined] | | | |
| RF | | p = 0.000 | p = 0.000 | p = 0.000 |
| | | (RF) [combined] | (RF) [combined] | (RF) [combined] |
| | | | p = 0.000 | |
| | | | (RF) [PAT] | |
| SVM | | | NS | NS |
| ANN | | | | p = 0.000 |
| | | | | (ANN) [combined] |

Systolic

| | | | | |
|---|---|---|---|---|
| Lasso | p = 0.000 | NS | NS | p = 0.000 |
| | (RF) [combined] | | | (Lasso) [combined] |
| RF | | p = 0.000 | p = 0.000 | p = 0.000 |
| | | (RF) [combined] | (RF) [combined] | (RF) [combined] |
| | | | p = 0.000 | |
| | | | (RF) [PAT] | |
| SVM | | | p = 0.002 | p = 0.000 |
| | | | (SVM) [morphology] | (SVM) [combined] |
| ANN | | | | p = 0.000 |
| | | | | (ANN) [combined] |

Diastolic

| | | | | |
|---|---|---|---|---|
| Lasso | p = 0.000 | NS | NS | NS |
| | (RF) [combined] | | | |
| RF | | p = 0.000 | p = 0.000 | p = 0.000 |
| | | (SVM) | (RF) [combined] | (RF) [combined] |
| | | [combined] | p = 0.000 | |
| | | | (RF) [PAT] | |
| SVM | | | NS | NS |
| ANN | | | | NS |

*denotes a significant difference using the Bonferroni-Holm met; NS indicates not significant.
Data are shown as p values.
Parentheses denote the method with the significantly lower mean error.
Square parentheses denote the feature dataset.

TABLE 4 presents the statistical comparison results for the mean errors. While several significant differences were reported, all model-feature dataset combinations met the standard for bias or mean error. Hence, each poorer performing combination still exhibited an acceptable error. TABLE 6 presents the statistical comparison results for the absolute errors (which are related to the error standard deviations).

For diastolic BP, TABLE 7 shows that SVM performed statistically better than RF and ANN, while RF performed better than ANN based on absolute errors. However, TABLE 3 shows that the actual SD difference was <2 mm Hg between these methods for diastolic BP using the combined features dataset—and the corresponding SD difference between RF and SVM was only 0.21 mm Hg. Overall, for diastolic BP estimation, the results seem to find these two methods somewhat equivalent in performance. For MAP and systolic BP, RF performed significantly better than SVM and ANN based on absolute errors (standard deviation).

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

### DEFINITIONS

As used herein, “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

As used herein, a “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

As used herein, when a quantitative characteristic (e.g., largest lateral dimension) is described as “in a range of,” when accompanied by a smaller value and a larger value, this refers to the quantitative characteristic having a value between the smaller value and the larger value or equal to the smaller value of the larger value.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component or directly connected to the second component. As used herein, the term “plurality” denotes two or more. For example, a plurality of components indicates two or more components.

The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

As used herein, specific details are given to provide a thorough understanding of the examples. However, it will be understood by one of ordinary skill in the art that the examples may be practiced without these specific details. For example, electrical components/devices may be shown in block diagrams in order not to obscure the examples in unnecessary detail. In other instances, such components, other structures and techniques may be shown in detail to further explain the examples.

It is also noted that the examples may be described as a process, which is depicted as a flowchart, a flow diagram, a finite state diagram, a structure diagram, or a block diagram. Although a flowchart may describe the operations as a sequential process, many of the operations can be performed in parallel, or concurrently, and the process can be repeated. In addition, the order of the operations may be re-arranged. A process is terminated when its operations are completed. A process may correspond to a method, a function, a procedure, a subroutine, a subprogram, etc. When a process corresponds to a software function, its termination corresponds to a return of the function to the calling function or the main function.

The previous description of the disclosed implementations is provided to enable any person skilled in the art to make or use the present invention. Various modifications to these implementations will be readily apparent to those skilled in the art, and the generic principles defined herein may be applied to other implementations without departing from the spirit or scope of the invention. Thus, the present invention is not intended to be limited to the implementations shown herein but is to be accorded the widest scope consistent with the principles and novel features disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

1. A system for estimating blood pressure comprising:
at least one sensor configured to measure at least one waveform related to blood pressure from a patient;
at least one processor; and
at least one computer-readable storage medium having encoded thereon executable instructions that, when executed by the at least one processor, cause the at least one processor to carry out a method comprising:
a) receiving, from the at least one sensor, the at least one waveform and determining, from the at least one waveform, one or more features comprising one or more temporal features or one or more morphology features;
b) analyzing the one or more features using one or more trained models to determine one or more blood pressure (BP) value associated with the patient; and
c) outputting the one or more BP value determined for the patient based on the received one or more features.

2. The system of claim 1, further comprising a user interface.

3. The system of claim 1, wherein at least one of the at least one sensor, the at least one processor, and the at least one computer-readable storage medium is wearable.

4. The system of claim 1, wherein the at least one sensor comprises a photoplethysmography (PPG) sensor or an electrocardiogram (ECG) sensor.

5. The system of claim 1, wherein each of the one or more BP value comprises one or more of a systolic BP, a diastolic BP, and/or a mean arterial pressure (MAP).

6. The system of claim 1, wherein the received one or more features is a preprocessed feature set, the preprocessed feature set comprising the one or more features configured as a function of one or more of: a pre-ejection period, a square of pulse transit time, a PPG intensity ratio, and/or a Womersly number.

7. The system of claim 1, wherein the one or more trained models comprises one or more of: a lasso model, a random forest model, a support vector machine model, an artificial neural network model, a long short term memory model, a RESNET deep learning model, and/or a combination or ensemble thereof.

8. The system of claim 1, wherein the analyzing comprises tuning hyperparameters for each of the one or more trained models.

9. The system of claim 1, wherein the one or more morphology features comprise: a BP cycle time, an ejection time, an artery fill time, an artery emptying time, a peak volume, a systolic volume, a systolic volume differential, a diastolic volume, a diastolic volume differential, or a combination thereof.

10. The system of claim 1, wherein the one or more morphology features are obtained from at least one PPG waveform, wherein the at least one PPG waveform is collected noninvasively over a radial artery.

11. The system of claim 1, wherein the one or more features further comprises biometric data, wherein the biometric data comprises one or more of: a pre-existing condition, an age, a weight, a height, a waist size, a body mass index (BMI), a sex, and/or a combination thereof.

12. The system of claim 1, wherein the one or more temporal features comprises one or more of: a pulse arrival time (PAT), a pulse transit time (PTT), a pulse rate, and/or a combination thereof.

13. The system of claim 12, wherein the PAT is based on a time difference between a peak of an ECG-R wave and a peak of a PPG waveform.

14. The system of claim 12, wherein the PTT is based on a difference between at least two PPG waveforms.

15. The system of claim 1, wherein the one or more temporal features and or the one or more morphology features are extracted from: a first PPG sensor a second PPG sensor, a first PPG sensor and a first ECG sensor, or a first ECG sensor and a second ECG sensor.

16. The system of claim 1, wherein each of the one or more trained models are trained using at least training temporal feature data and training morphology data from a plurality of prior patients.

17. The system of claim 16, wherein each of the prior patients has a pre-existing condition.

18. The system of claim 1, wherein the determining comprises selecting the one or more trained models from a plurality of models, wherein the selecting is based on evaluating a performance of the one or more trained models as compared to others of the plurality of models.

19. The system of claim 18, wherein the evaluating is based on one or more of an average error bias and/or a standard deviation of each of the plurality of models.

20. The system of claim 18, wherein the evaluating is performed independently for each of the one or more BP value.

21. The system of claim 1, wherein the determining comprises selecting an ensemble of two or more of the one or more trained models, wherein the selecting is based on evaluating a performance of the one or more trained models as compared to others of the one or more trained models.

22. The system of claim 21, wherein the ensemble comprises the two or more of the one or more trained models being in parallel or serial.

23. A method for estimating blood pressure, the method comprising:
a) determining, based on a received feature set of one or more features from a patient, one or more blood pressure (BP) value associated with the patient, the one or more features comprising one or more temporal features and or one or more morphology features, the determining comprising analyzing the one or more features using one or more trained models, wherein each of the one or more trained models are trained using at least training temporal feature data and training morphology data from a plurality of prior patients; and
b) outputting the one or more BP value determined for the patient based on the received feature set.

24-40. (canceled)

41. At least one storage medium having encoded thereon executable instructions that, when executed by at least one processor, cause the at least one processor to carry out a method comprising:
a) determining, based on a received feature set of one or more features from a patient, one or more blood pressure (BP) value associated with the patient, the one or more features comprising one or more temporal features or one or more morphology features, the determining comprising analyzing the one or more features using one or more trained models, wherein each of the one or more trained models are trained using at least training temporal feature data and training morphology data from a plurality of prior patients; and
b) outputting the one or more BP value determined for the patient based on the received feature set.

42-58. (canceled)

59. A system for estimating blood pressure comprising:
at least one processor; and
at least one computer-readable storage medium having encoded thereon executable instructions that, when executed by the at least one processor, cause the at least one processor to carry out a method comprising: determining, based on a received feature set of one or more features from a patient, one or more blood pressure (BP) value associated with the patient, the one or more features comprising one or more temporal features or one or more morphology features, the determining comprising analyzing the one or more features using one or more trained models, wherein each of the one or more trained models are trained using at least training temporal feature data and training morphology data from a plurality of prior patients.

60-81. (canceled)
